SEQUENCE LISTING (38 sequences)

SEQ ID NO: 1 - RNA, 21 nt, synthetic construct
  source 1..21: mol_type = other RNA
  misc_feature 1..21: note = standard hairpin sequence for lbaCas12a
taatttctactaagtgtagat

SEQ ID NO: 2 - DNA, 44 bp, synthetic construct
  source 1..44: mol_type = other DNA
cctacctagccagtccctatcagtgatagagataaacctcacac

SEQ ID NO: 3 - DNA, 44 bp, synthetic construct
  source 1..44: mol_type = other DNA
gtgtgaggtttatctctatcactgatagggactggctaggtagg

SEQ ID NO: 4 - DNA, 19 bp, synthetic construct
  source 1..19: mol_type = other DNA
tctctatcactgataggga

SEQ ID NO: 5 - DNA, 44 bp, synthetic construct
  source 1..44: mol_type = other DNA
cctacctacctcgccttgttatccgctcacaataaacctcacac

SEQ ID NO: 6 - DNA, 44 bp, synthetic construct
  source 1..44: mol_type = other DNA
gtgtgaggtttattgtgagcggataacaaggcgaggtaggtagg

SEQ ID NO: 7 - DNA, 17 bp, synthetic construct
  source 1..17: mol_type = other DNA
ttgtgagcggataacaa

SEQ ID NO: 8 - DNA, 49 bp, synthetic construct
  source 1..49: mol_type = other DNA
cctacctaaccttaacgatacggtacgtttcgtatcataaacctcacac

SEQ ID NO: 9 - DNA, 49 bp, synthetic construct
  source 1..49: mol_type = other DNA
gtgtgaggtttatgatacgaaacgtaccgtatcgttaaggttaggtagg

SEQ ID NO: 10 - DNA, 30 bp, synthetic construct
  source 1..30: mol_type = other DNA
atgatacgaaacgtaccgtatcgttaaggt

SEQ ID NO: 11 - DNA, 52 bp, synthetic construct
  source 1..52: mol_type = other DNA
cctacctaataatacaaacagaccagattgtctgtttgtttaaacctcacac

SEQ ID NO: 12 - DNA, 52 bp, synthetic construct
  source 1..52: mol_type = other DNA
gtgtgaggtttaaacaaacagacaatctggtctgtttgtattattaggtagg

SEQ ID NO: 13 - DNA, 32 bp, synthetic construct
  source 1..32: mol_type = other DNA
aacaaacagacaatctggtctgtttgtattat

SEQ ID NO: 14 - DNA, 44 bp, synthetic construct
  source 1..44: mol_type = other DNA
cctacctaccattgattggacgttcaatataataaacctcacac

SEQ ID NO: 15 - DNA, 44 bp, synthetic construct
  source 1..44: mol_type = other DNA
gtgtgaggtttattatattgaacgtccaatcaatggtaggtagg

SEQ ID NO: 16 - DNA, 22 bp, synthetic construct
  source 1..22: mol_type = other DNA
attgattggacgttcaatataa

SEQ ID NO: 17 - DNA, 57 bp, synthetic construct
  source 1..57: mol_type = other DNA
cctacctagattccacctaaatgtaacagtcacgtcggttatattcaaacctcacac

SEQ ID NO: 18 - DNA, 57 bp, synthetic construct
  source 1..57: mol_type = other DNA
gtgtgaggtttgaatataaccgacgtgactgttacatttaggtggaatctaggtagg

SEQ ID NO: 19 - DNA, 38 bp, synthetic construct
  source 1..38: mol_type = other DNA
aaatgaatataaccgacgtgactgttacatttaggtgg

SEQ ID NO: 20 - RNA, 69 nt, synthetic construct
  source 1..69: mol_type = other RNA
  regulatory 1..24: regulatory_class = promoter
  stem_loop 24..45: note = Cas12a hairpin
gcgctaatacgactcactatagggtaatttctactaagtgtagattctctatcactgatagggactggc

SEQ ID NO: 21 - RNA, 69 nt, synthetic construct
  source 1..69: mol_type = other RNA
  regulatory 1..24: regulatory_class = promoter
  stem_loop 25..45: note = Cas12a hairpin
gcgctaatacgactcactatagggtaatttctactaagtgtagatttgtgagcggataacaaggcgagg

SEQ ID NO: 22 - RNA, 69 nt, synthetic construct
  source 1..69: mol_type = other RNA
  regulatory 1..24: regulatory_class = promoter
  stem_loop 25..45: note = Cas12a hairpin
gcgctaatacgactcactatagggtaatttctactaagtgtagattgatacgaaacgtaccgtatcgtt

SEQ ID NO: 23 - RNA, 69 nt, synthetic construct
  source 1..69: mol_type = other RNA
  regulatory 1..24: regulatory_class = promoter
  stem_loop 25..45: note = Cas12a hairpin
gcgctaatacgactcactatagggtaatttctactaagtgtagataacaaacagacaatctggtctgtt

SEQ ID NO: 24 - RNA, 69 nt, synthetic construct
  source 1..69: mol_type = other RNA
  regulatory 1..24: regulatory_class = promoter
  stem_loop 25..45: note = Cas12a hairpin
gcgctaatacgactcactatagggtaatttctactaagtgtagatttatattgaacgtccaatcaatgg

SEQ ID NO: 25 - RNA, 69 nt, synthetic construct
  source 1..69: mol_type = other RNA
  regulatory 1..24: regulatory_class = promoter
  stem_loop 25..45: note = Cas12a hairpin
gcgctaatacgactcactatagggtaatttctactaagtgtagataatataaccgacgtgactgttaca

SEQ ID NO: 26 - DNA, 624 bp, synthetic construct
  source 1..624: mol_type = other DNA
atgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcctaa

SEQ ID NO: 27 - DNA, 1083 bp, synthetic construct
  source 1..1083: mol_type = other DNA
atgaaaccagtaacgttatacgatgtcgcagagtatgccggtgtctcttatatgaccgtttcccgcgtggtgaaccaggccagccacgtttctgcgaaaacgcgggaaaaagtggaagcggcgatggtggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattatttactcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 28 - DNA, 606 bp, synthetic construct
  source 1..606: mol_type = other DNA
atggcacgtaccccgagccgtagcagcattggtagcctgcgtagtccgcatacccataaagcaattctgaccagcaccattgaaatcctgaaagaatgtggttatagcggtctgagcattgaaagcgtggcacgtcgcgccggtgcaggcaaaccgaccatttatcgttggtggaccaacaaagcagcactgattgccgaagtgtatgaaaatgaaatcgaacaggtacgtaaatttccggatttgggtagctttaaagccgatctggattttctgctgcataatctgtggaaagtttggcgtgaaaccatttgtggtgaagcatttcgttgtgttattgcagaagcacagttggaccctgtaaccctgacccaactgaaagatcagtttatggaacgtcgtcgtgagataccgaaaaaactggttgaagatgccattagcaatggtgaactgccgaaagatatcaatcgtgaactgctgctggatatgatttttggtttttgttggtatcgcctgctgaccgaacagttgaccgttgaacaggatattgaagaatttaccttcctgctgattaatggtgtttgtccgggtacacagtgttgataa

SEQ ID NO: 29 - DNA, 612 bp, synthetic construct
  source 1..612: mol_type = other DNA
atgagcccgaaacgtcgtacccaggcagaacgtgcaatggaaacccagggtaaactgattgcagcagcactgggtgttctgcgtgaaaaaggttatgcaggttttcgtattgcagatgttccgggtgcagccggtgttagccgtggtgcacagagccatcattttccgaccaaactggaactgctgctggcaacctttgaatggctgtatgagcagattaccgaacgtagccgtgcacgtctggcaaaactgaaaccggaagatgatgttattcagcagatgctggatgatgcagcagaattttttctggatgatgattttagcatcggcctggatctgattgttgcagcagatcgtgatccggcactgcgtgaaggtattcagcgtaccgttgaacgtaatcgttttgttgttgaagatatgtggctgggtgtgctggtgagccgtggtctgagccgtgatgatgccgaagatattctgtggctgatttttaacagcgttcgtggtctggtagttcgtagcctgtggcagaaagataaagaacgttttgaacgtgtgcgtaatagcaccctggaaattgcacgtgaacgttatgcaaaattcaaacgttga

SEQ ID NO: 30 - DNA, 588 bp, synthetic construct
  source 1..588: mol_type = other DNA
atgccgaaactgggtatgcagagcattcgtcgtcgtcagctgattgatgcaaccctggaagcaattaatgaagttggtatgcatgatgcaaccattgcacagattgcacgtcgtgccggtgttagcaccggtattattagccattatttccgcgataaaaacggtctactggaagcaaccatgcgtgatattaccagccagctgcgtgatgcagttctgaatcgtctgcatgcactgccgcagggtagcgcagaacagcgtctgcaggcaattgttggtggtaattttgatgaaacccaggttagcagcgcagcaatgaaagcatggctggcattttgggcaatcagcatgcatcagccgatgctgtatcgtctgcagcaggttagcagtcgtcgtctgctgagcaatctggttagcgaatttcgtcgtgaactgcctcgtgaacaggcacaagaggcaggttatggtctggcagcactgattgatggtctgtggctgcgtgcagcactgagcggtaaaccgctggataaaacccgtgcaaatagcctgacccgtcattttatcacccagcatctgccgaccgattga

SEQ ID NO: 31 - DNA, 569 bp, synthetic construct
  source 1..569: mol_type = other DNA
tataccatgccgcgtccgaaattaaaatcggatgacgaagttcttgaggcagcgactgtagtattgaaacgctgtggtcccattgagtttacgctttctggggtcgcgaaagaagttggtcttagccgcgcagcgttgatccaacgttttaccaaccgcgacacgctgttggtgcgtatgatggaacgtggcgtggagcaggtgcgtcactacttaaatgcgatccccattggggcgggccctcagggtttatgggagtttcttcaagttttagtgcgtagcatgaatacgcgtaatgacttctctgtaaactacttgatttcgtggtacgagcttcaagtccccgaattgcgcacactggcgattcaacgcaaccgcgcagtagtagaaggtatccgcaagcgtttgcctcctggggcccctgcggcggctgagttgctgctgcatagcgtgattgcgggtgccacgatgcagtgggcggtggacccggacggtgaattagccgatcatgtattagcccagatcgctgcgatcttgtgtttaatgttccccgaacatgatgactttca

SEQ ID NO: 32 - DNA, 28 bp, synthetic construct
  source 1..28: mol_type = other DNA
gtaacagtcacgtcggttatattctaaa

SEQ ID NO: 33 - DNA, 28 bp, synthetic construct
  source 1..28: mol_type = other DNA
cattgtcagtgcagccaatataagattt

SEQ ID NO: 34 - DNA, 27 bp, synthetic construct
  source 1..27: mol_type = other DNA
gtaacagtcacgtcggttatattcaaa

SEQ ID NO: 35 - DNA, 27 bp, synthetic construct
  source 1..27: mol_type = other DNA
cattgtcagtgcagccaatataagttt

SEQ ID NO: 36 - DNA, 30 bp, synthetic construct
  source 1..30: mol_type = other DNA
gtaacagtcacgtcggttatattcgctaaa

SEQ ID NO: 37 - DNA, 28 bp, synthetic construct
  source 1..28: mol_type = other DNA
cattgtcagtgcagccaatataaggttt

SEQ ID NO: 38 - protein, 10 aa, synthetic construct
TTTTTTTTTT

(19) United States
(12) Patent Application Publication
(10) Pub. No.: US 2024/0094229 A1
(43) Pub. Date: Mar. 21, 2024

(54) CRISPR-CAS /TRANSCRIPTION FACTOR-BASED COMPETITION ASSAY FOR DETECTION OF MOLECULAR ANALYTES

(71) Applicant: Arizona Board of Regents on Behalf of Arizona State University, Scottsdale, AZ (US)

(72) Inventors: Alexander Green, Chestnut Hill, MA (US); Soma Chaudhary, Tempe, AZ (US); Kirstie Swingle, Henrico, VA (US)

(21) Appl. No.: 18/463,628

(22) Filed: Sep. 8, 2023

Related U.S. Application Data
(60) Provisional application No. 63/404,847, filed on Sep. 8, 2022.

(51) Int. Cl. G01N 33/94; C12Q 1/6823; G01N 33/53; G01N 33/542; G01N 33/543

(52) U.S. Cl. CPC G01N 33/9446; C12Q 1/6823; G01N 33/5308; G01N 33/542; G01N 33/54388

(57) ABSTRACT

Provided herein are highly modular and sensitive biosensors for small molecule detection using CRISPR/C as enzymes having collateral single-stranded nucleic acid cleavage activity. Also provided herein are uses of the biosensors in detection platforms for convenient, low-cost assessment of safety and purity of consumable samples, environmental samples, and agricultural products.

### CROSS-REFERENCE TO RELATED APPLICATIONS

This application claims the benefit of U.S. Provisional Application No. 63/404,847, filed Sep. 8, 2022, the content of which is incorporated herein by reference in its entirety.

### STATEMENT REGARDING FEDERALLY FUNDED RESEARCH OR DEVELOPMENT

Not applicable.

### SEQUENCE LISTING STATEMENT

This application includes a sequence listing in XML format named “112624 01420 SL ST26.xml” which is 44,188 bytes in size and was created on Nov. 6, 2023. The sequence listing is electronically submitted via Patent Center with the application and is incorporated herein by reference in its entirety.

### BACKGROUND

The use of antibiotics in agriculture and aquaculture are on the rise and the effects on the surrounding environments are a growing health hazard (35). For example, certain species of bacteria are showing resistance to common antibiotics like erythromycin found in aquaculture, and this resistance can be gene-transferred to different species (36). Other examples include antibiotics found in runoff and ground water from farms and pig slurry which can also lead to antibiotic resistance (37). All these factors lead to potential health issues, and having easy and inexpensive antibiotic detection devices would aid in understanding and preventing further problems.

Small-molecule/transcription factor pairs are ubiquitous throughout nature and enable cells to tightly control gene expression in response to changing concentrations of molecules in the environment. Allosteric transcription factors regulate gene expression by binding to specific DNA operator sites to promote or inhibit transcription in response to surrounding cognate small molecules. Transcription factors (TF) respond to a wide range of molecules including small molecules and drugs like antibiotics. This prevalence throughout nature, wide target range, and ability to interact with DNA make small molecule/TF pairs excellent targets for biosensor development.

Synthetic biology approaches have been used to build modular and sensitive TF-based biosensors to detect small molecules including drugs. Many of these approaches rely on TF/enzyme competition of DNA binding domains, where a measurable readout is given upon enzyme binding to the DNA in vitro (1-5). Other approaches include whole cell biosensors (6, 7), CRISPR/Cas12a based detection that uses TFs (8), protein detection (9), aptamers (10-12) and Cas13a based detection (13). With the growing issue of small molecule contaminants like antibiotics in water sources (14, 15), easy to use and sensitive biosensors are essential and can be constructed using TF-based systems. However, current issues with the methods given above include the use of expensive instruments like fluorescence plate readers, fluorescent rather than visible reporters, biosafety concerns, poor robustness and biosafety concerns with whole cell biosensors, and relatively high detection limits, often in the μM range.

CRISPR-based biosensors have gained popularity in biosensor development with the ease of target programmability and the utilization of collateral cleavage capabilities from certain Cas enzyme types (16-18). Enzymes like Cas12a target a specific DNA sequence based on the preprogrammed guide RNA (gRNA). Upon recognition, the enzyme is activated and cleaves the target DNA, and through the collateral cleavage mechanism, begins to non-specifically cleave single-stranded DNA (ssDNA) molecules present in the reaction. By using ssDNA with chemical modifications, the non-specific collateral cleavage can be used as a measurable readout. CRISPR-based biosensors offer benefits including built-in signal amplification, easy target programming by changing the gRNA sequence, and readout control through ssDNA modifications. With the readout control, CRISPR based biosensors can be employed on paper-based devices, resulting in field deployable devices (19-26).

Several small-molecule detection methods have utilized CRISPR-Cas biosensor capabilities for the construction of more modular and sensitive systems. Many of these biosensors use aptamers to activate the Cas12a enzyme and typically result in nM detection limits (10-12). These methods merge the benefits of CRISPR based diagnostics with TF-small molecule detection, but are limited in small molecule targets when requiring a corresponding aptamer, and in field-deployable options when instruments such as centrifuges are required to separate free DNA from TF-bound beads. Therefore, improved systems for conveniently and rapidly detecting small molecules in the field are needed.

### SUMMARY OF THE INVENTION

Disclosed herein are methods for detecting a target molecule in a sample using CRISPR/Cas enzymes having collateral single-stranded nucleic acid cleavage activity. The method comprises: (a) contacting to the sample a synthetic double-stranded DNA (dsDNA) substrate comprising an operator sequence specific to a binding protein, and a protospacer adjacent motif (PAM) sequence; a binding protein; a Cas nuclease that exhibits collateral single-stranded DNase (ssDNase) activity; a guide RNA (gRNA), wherein the gRNA hybridizes the reverse complement of the operator sequence or the operator sequence; and a single-stranded DNA (ssDNA) reporter construct comprising a detectable tag, wherein, in the presence of the target molecule, the target molecule binds to the binding protein, the gRNA forms a complex with the PAM sequence, and the Cas nuclease cleaves the ssDNA reporter construct; and (b) detecting cleavage of the ssDNA reporter construct, thereby detecting the target molecule in the sample.

Also disclosed herein is a biosensor device comprising: a plurality of synthetic dsDNA substrates, each substrate comprising an operator sequence specific to a different binding protein, and a protospacer adjacent motif (PAM) sequence; a plurality of binding proteins, wherein each binding protein binds to one of the operator sequences; a ssDNA reporter construct comprising a detectable tag; a Cas nuclease that exhibits collateral ssDNase activity; and a plurality of gRNAs, wherein each gRNA hybridizes the reverse complement of the operator sequence or the operator sequence.

In some embodiments, the gRNA comprises the reverse complement of the operator sequence, or comprises the operator sequence, and binds to the PAM sequence. Also disclosed herein is a kit comprising the biosensor device described herein.

### BRIEF DESCRIPTION OF THE DRAWINGS

FIG. 1 is a schematic illustrating an exemplary small molecule biosensor. (Step a) Transcription factors bind to dsDNA target, blocking binding of the Cas12a/gRNA complex. (Step b) When small molecules are added, the transcription factor releases the dsDNA, enabling binding by Cas12a/gRNA. (Step c) Recognition of the dsDNA target activates Cas12a resulting in collateral cleavage of reporter ssDNA. (Step d) Reporter cleavage is monitored on lateral flow strips or via fluorescence.

FIGS. 2A-2B is a fluorescence study illustrating that TetO sequence-based dsDNA substrate can activate Cas12a/gRNA complex. FIG. 2A shows concentrations between 1-50 nM dsDNA substrate show fluorescence signal, indicating activation of Cas12a/gRNA. FIG. 2B shows different ratios and concentrations of Cas12a to gRNA tested. For fluorescence studies, 53 nM Cas12a and 100 nM gRNA were used. For lateral flow assays, 106 nM Cas12a and 100 nM gRNA were used.

FIGS. 3A-3F illustrate transcription factor mediated detection of aTc and Doxy using Cas12a. FIG. 3A shows TetR represses Cas12a activation. n=4 technical replicates, bars represent the mean±s.d. FIG. 3B shows effects of protein concentration of Cas12a signal with aTc introduced. FIGS. 3C and 3D show fluorescence curves monitoring Cas12a cleavage with the addition of aTc and Doxy. FIGS. 3E and 3F show a titration of small molecule with 10 μM TetR.

FIGS. 4A-4C illustrate DNA modifications and sensor specificity. FIG. 4A show the different types of spacers tested for MphR, CymR, and PhiF. (SEQ ID NOs. 32, 33, 34, 35, 36, 33, 32, 37, top to bottom). FIG. 4B illustrates the on/off ratios, which were calculated by dividing the target only sample by the target+TF sample. The data was normalized to the maximum output for each set. FIG. 4C shows orthogonality data as fluorescence endpoint values from 1 hour of cleavage when exposed to each small molecule using all transcription factors. Data were normalized by setting the correct target SM signal to 100.

FIGS. 5A-5H show fluorescence curves for the detection of (5A) Erythromycin, (5B) Clarithromycin, (5C) Azithromycin, (5D) Roxithromycin, (5E) Cuminic Acid, (5F) DAPG (5G) IPTG, and (5H) Choline chloride and with their corresponding transcription factors.

FIGS. 6A-6E shows dose response curves for (5A) IPTG, (5B) Erythromycin, (5C) Cuminic Acid, (5D) DAPG, and (5E) Choline chloride. All samples were done in replicates of 4. Standard deviation bars are plotted. Welch's t-test was used to determine statistical differences in SM+/−samples. (P-value<0.001 ***, P<0.01**).

FIGS. 7A-7F illustrate sensor tunability by altering protein/DNA concentrations and gRNA length. n=4 technical replicates, mean values are plotted±s.d., Welch's t-test was used to determine statistical significance for each sample compared to the reaction without the small molecule analyte; **P<0.01, ***P<0.001. FIG. 7A-B shows dose response curves for 2, 10, 20 μM TetR with a titration of aTc (7A) and Doxy (7B). FIG. 7C-D shows dose response curves for 50× TetR starting with 5 nM and 50 nM DNA with a titration of aTc (7C) and Doxy (7D). Results are shown at 1 hr. endpoint. FIG. 7E-F shows dose response curves with 20 nt gRNA for a titration of aTc (7E) and Doxy (7F). Results are shown after 30 minutes of cleavage reaction.

FIGS. 8A-8C illustrate cleavage rate and signal as a function of gRNA length. FIG. 8A shows cleavage rates determined using linear regression to fit the fluorescence data over a ten-minute period representing the steepest increase in fluorescence over time. The positive sample contains 20 μM aTc. FIG. 8B. shows on/off ratios calculated by dividing the mean analyte positive sample by the mean analyte negative sample. FIG. 8C shows a plot of on/off values for gRNA length for MphR. 20 nt. and 24 nt. long gRNA were tested for the pTTT* MphR set. Erythromycin was titrated and on/off ratios were calculated by dividing the mean analyte positive sample by the mean analyte negative sample.

FIGS. 9A-9C illustrate a quantitative analysis for small molecule detection. FIG. 9A shows the rate of cleavage was determined by calculating the slope of kinetic curves corresponding to Cas12a activation induced by a titration of small molecule (cuminic acid) using the CymR biosensor. FIG. 9B shows a plot of the corresponding slopes and SM concentrations. FIG. 9C shows a calibration curve for determining SM concentration of unknown samples using the cleavage rate. The linear region, 50 μM and below, were fit to generate the calibration curve. This method can be used to generate calibration curves for any of the SM biosensors described herein.

FIGS. 10A-10D illustrate lateral flow readout for small molecule detection. FIG. 10A is a schematic of lateral flow. A FAM-biotin modified reporter was used for LF readout. Binding to only the control line indicates a negative test. Binding to both control and test lines indicate a positive test. FIG. 10B shows a raw image of TetR lateral flow readout. FIG. 10C shows a plot of the mean pixel intensity from FIG. 10B, normalized to aTc maximum signal. FIG. 10D shows a limit of detection for aTc and Doxy using LF readout. Plots show triplicate means with error bars representing the standard deviation. EC 50 is calculated using dose response fitting from OriginLab.

FIGS. 11A-11B show lateral flow readouts illustrating lateral flow optimization. FIG. 11A shows reporter concentration. FIG. 11B shows cleavage reaction times.

FIGS. 12A-12B shows a quantitative analysis developed using the Matlab function imread to read in the pixel values for the lateral flow images. RGB value means for each assay were plotted. The peak height of the second band was used to represent band intensity. Error! Reference source not found. analysis for two different concentrations of Doxy is shown, where 12A represents a strong signal and 12B represents a low signal.

FIG. 13 shows replicates of the lateral flow assay shown in FIG. 10.

FIGS. 14A-14B illustrate erythromycin detection using lateral flow. FIG. 14A shows erythromycin concentrations from 0.05-20 μM tested on lateral flow. FIG. 14B shows mean pixel intensity for the four lowest concentrations and negative sample.

FIG. 15 shows lateral flow mean pixel intensity values for Lad based detection of IPTG. Purple bars (pos) show IPTG positive samples for 10 and 20 μM Lad. Blue bars (neg) show the IPTG negative samples.

FIGS. 16A-16B illustrate detection of doxycycline using different water sources. Doxycycline was added to ultrapure water, ground water, wastewater, and tap water to analyze the effects of water sources on SM detection. FIG. 16A shows the on/off ratios of Cas12a cleavage for each sample at 0.05-1 μM Doxycycline. All samples show high performance at 100 nM except for ground water. FIG. 16B shows the limit of detection for each water source. EC 50 values could not be calculated but all 4 samples showed a detection limit of 50 nM being 3 times the standard deviation of the small molecule negative sample. n=4.

FIG. 17 shows a dose response fitting.

FIG. 18 shows a toxicity study for DMSO. The toxicity of solvents on Cas12a activation was tested by titrating in DMSO and monitoring the fluorescence output over two hours. A more significant decrease in activation was observed above 1% DMSO. The highest concentration of aTc for the limit of detection experiments contained 0.059% DMSO for 40 μM, following 0.03% for 20 μM aTc.

### DETAILED DESCRIPTION

All publications, including but not limited to patents and patent applications, cited in this specification are herein incorporated by reference as though set forth in their entirety in the present application.

Provided herein are CRISPR-based biosensors and methods for detecting small molecules with high modularity and sensitivity. The biosensors uniquely use protein competition between a Cas enzyme and a DNA binding protein for a specific DNA sequence, and activation/deactivation depends on which protein is bound to the DNA. This is directly affected by the presence of target analytes and can be visualized on both a fluorescence plate reader and a lateral flow assay. The sensors can detect small molecule antibiotics such as tetracycline, doxycycline, and erythromycin, as well as other small molecules such as isopropyl β-D-1-thiogalactopyranoside (IPTG), 2,4-Diacetylphloroglucinol (DAPG), and cuminic acid. Overall, these biosensors offer a wide range of advantages. They can detect various small molecules, they offer tunability, and they can be applied to paper-based systems, including without limitation, lateral flow assays, paper-based microfluidic systems, paper-based cell-free transcription/translation reactions, and paper-based transcription reactions that generate a visible or fluorescent signal. The biosensors have also proven functional in different types of water including tap water and wastewater.

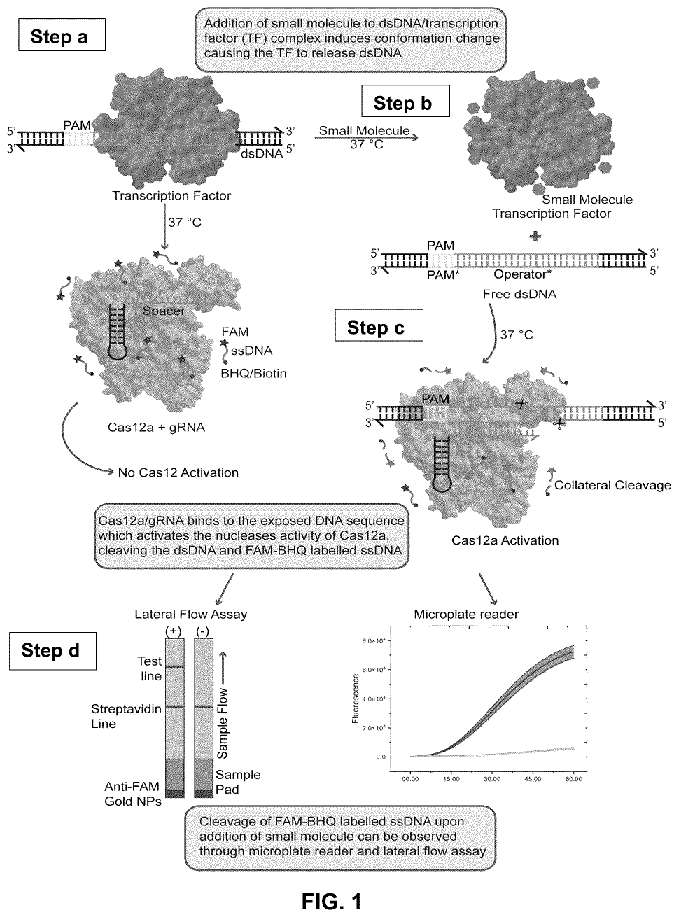

Accordingly, in a first aspect, provided herein is a biosensor for detecting a target molecule or analyte of interest. The general schematic of the biosensor is illustrated in FIG. 1. The biosensor employs a CRISPR-Cas system in a competition assay for a target DNA sequence. In some embodiments, the target DNA sequence includes an operator sequence or a eukaryotic regulatory sequence. Clustered Regularly Interspaced Short Palindromic Repeats (CRISPR)-Cas systems function as a prokaryotic adaptive immune defense through the recognition and degradation of invading viral RNA/DNA. These systems use guide RNAs (gRNAs) that direct Cas enzymes to cleave targeted DNA and RNA. CRISPR-Cas systems recognize target DNA that contains a region complementary to the gRNAs along with a T-rich protospacer adjacent motif (PAM). Upon recognition, Cas nuclease activity is initiated and a staggered dsDNA break is fashioned after the PAM site. The CRISPR-Cas system employed in the sensors and methods described herein utilizes Cas enzymes that additionally exhibit collateral cleavage activity on surrounding single-stranded DNA (ssDNA) molecules.

Thus, the biosensor includes a gRNA and a Cas nuclease that exhibits collateral ssDNase activity. The biosensor further includes a synthetic double-stranded DNA (dsDNA) substrate, selected based on a corresponding DNA binding protein for which the target analyte serves as a ligand. The biosensor further includes said DNA binding protein. The gRNA hybridizes the dsDNA substrate (e.g., is complementary to, or shares regions of complementarity with the dsDNA), and therefore competes with the DNA binding protein. When the target analyte is present in the sample, the analyte binds the DNA protein, thereby releasing it from the substrate.

This frees the substrate for gRNA-Cas binding. The biosensor additionally includes a ssDNA reporter construct. Upon gRNA-Cas binding to the substrate, the collateral cleavage activity of the Cas enzyme cleaves the surrounding ssDNA reporters. Therefore, presence of the target analyte in the sample is determined by signal generated from cleavage of the ssDNA reporters.

In some embodiments, the gRNA comprises the reverse complement of the operator sequence, or a region thereof, wherein the region of complementarity provides sufficient hybridization specificity or hybridization strength to outcompete the DNA binding protein. In some embodiments, the gRNA comprises the operator sequence or a region thereof, wherein the region of complementarity provides sufficient hybridization specificity or hybridization strength to outcompete the DNA binding protein. Accordingly, the gRNA spacer and operator sequence are not necessarily the same length. In some embodiments, the gRNA may have additional bases not found in the operator sequence. In some embodiments, the gRNA may comprise a sequence that base pairs with only a portion (a region) of the operator sequence. In some embodiments, the portion (region) is contiguous. In some embodiment the portion (region) is non-contiguous, and the gRNA comprises, for example, one or more mismatches relative to the operator sequence or the reverse complement of the operator sequence.

In some embodiments, the biosensor includes a gRNA and a Cas nuclease that exhibits ssRNase activity. The biosensor includes a ssDNA or RNA substrate, selected based on a corresponding RNA binding protein for which the target analyte serves as a ligand. The biosensor further includes said RNA binding protein. In some embodiments, the RNA binding protein is an allosteric RNA binding protein, where the protein's binding to an RNA sequence is modulated by the presence of a particular analyte, such as those described in Clingman et al. (38). The gRNA is complementary to the RNA substrate, and therefore competes with the RNA binding protein. When the target analyte is present in the sample, the analyte binds the RNA binding protein, thereby releasing it from the substrate. This frees the substrate for gRNA-Cas binding. The biosensor additionally includes a ssDNA or RNA reporter construct. Upon gRNA-Cas binding to the substrate, the collateral cleavage activity of the Cas enzyme cleaves the surrounding reporters. Therefore, presence of the target analyte in the sample is determined by signal generated from cleavage of the ssDNA/RNA reporters.

In some embodiments, the Cas enzyme is Cas12a. In some embodiments, the Cas12a is from Lachnospiraceae bacterium. Cas12a from other bacterial species may be used, such as acidaminococcus sp. and francisella novicida. It should be understood that similar systems can be implemented using other CRISPR/Cas enzymes that exhibit collateral ssDNase or ssRNase activity or are capable of multiple-turnover RNA or DNA cleavage reactions. For example, CRISPR enzymes that exhibit collateral ssDNase or ssRNase activity include, without limitation, Cas12a, Cas13a, Cas13b, Cas13d, Cas12g1, and Cas12i1.

In some embodiments, the ssDNA reporter construct comprises a detectable tag. The tag may include a small molecule. The small molecule may be incorporated at one or more of the 5′ end, the 3′ end, and within the construct such that there's a minimum of 5 nucleotides between the modifications. In some embodiments, the ssDNA reporter construct comprises a first small molecule and a second small molecule. The small molecules can be selected from biotin, digoxigenin, and FAM (fluorescein). Other detectable molecules that may be used according to the sensors and methods provided herein include, without limitation, digoxigenin, enzymatic reporters (e.g., β-galactosidase, alkaline phosphatase, DHFR, CAT), fluorescent or chemiluminescent reporters (e.g., GFP variants, mCherry, luciferase, e.g., luciferase derived from the firefly (Photinus pyrahs) or the sea pansy (Renilla reniformis) and mutants thereof), etc. In an embodiment a reporter construct is designed with a fluorophore attached to one end, and a quencher attached to the other end. When the ssDNA is intact, the fluorophore-quencher pair remain in close enough distance for Förster Resonance Energy Transfer to occur and fluorescence is quenched. Upon cleavage of the reporter, the quencher-fluorophore pair is separated, and fluorescence is induced. The presence or absence of the single-stranded reporter construct can also be detected using nucleic acid sensors, such as a toehold switch, loop-mediated riboregulator, three-way junction (3WJ) repressor, single-nucleotide-specific programmable riboregulator (SNIPR), toehold repressor, aptaswitch, etc., to produce a protein reporter signal that can be detected in cell-free transcription-translation reactions or in vitro transcription reactions. Potential reporter signals include enzymatic reporters (e.g., β-galactosidase, alkaline phosphatase, DHFR, CAT), fluorescent or chemiluminescent reporters (e.g., GFP variants, mCherry, luciferase, e.g., luciferase derived from the firefly (Photinus pyralis) or the sea pansy (Renilla reniformis) and mutants thereof), fluorogenic aptamer-dye combinations (e.g. Broccoli/DFHBI-1T, Corn/DFHO, Red Broccoli/DFHO, Mango IV/TO1-Biotin), etc. When the ssDNA reporter construct comprises biotin and fluorescein, nanoparticles comprising anti-FAM antibodies can be used to detect the cleavage event. For example, the assay can be done on a lateral flow device configured such that the Cas-ssDNA cleavage reaction occurs on a sample pad at one end of a lateral flow strip. Without the antibody (no activation), intact reporter ssDNAs are captured by the biotin line and in turn capture the nanoparticles via the anti-FAM antibodies. Thus, inactivated Cas reactions will yield both a sample line and a control line in the lateral flow strip. Upon recognition of a target molecule in a sample contacted to the lateral flow, the gRNA/Cas-nuclease complex is activated. Activation of Cas cleavage of the ssDNA reporters results in the formation of only a single band. In other words, activated Cas reactions will yield a control line in the lateral flow strip but no streptavidin sample line.

In some embodiments, the DNA binding protein is a transcription factor. The transcription factor may be selected from, for example, TetR, PhiF, CymR, Lad, MphR, and BetL. In some embodiments, the target molecule is an antibiotic. The target molecule may be selected from, for example, aTc, doxycycline, DAPG, cuminic acid, IPTG, clarithromycin, erythromycin, azithromycin, and roxithromycin. The biosensor can be broadly applied for any analyte that can bind to a recognition molecule that is conjugated to a nucleic acid and cause release of the recognition molecule from the nucleic acid upon binding. Thus, it can be used to detect molecular analytes such as antibiotics, sugars, and other small molecules, as well as combinations of analytes that bind to one another. The reactions can take place in vitro, in paper-based systems, and in living or fixed cells.

In some embodiments, different dsDNA configurations may be used. By way of example but not by way of limitation, in some embodiments, the fourth nucleotide of the PAM sequence can be incorporated into the operator sequence. In some embodiment, a mismatch may be introduced into the PAM sequence (e.g., a mutation in the PAM sequence). By way of example, a bulge may be introduced by including one, two or more nucleotides between the PAM sequence and operator on a single strand or including mismatches in both strands, thereby generating a bulge.

An advantage of this biosensor lies within the programmability of CRISPR/Cas12a target sequences. Instead of being confined to set specific binding sequences for DNA binding proteins, the gRNA sequence can be altered to match any needed sequence for Cas enzyme binding; this includes transcription factor binding domains. CRISPR/Cas systems have been employed in the detection of nucleic acids, small molecules, and more for this reason, but no CRISPR based small molecule detection currently exists with the same wide range application, simplistic design, and paper-based readout. For instance, the small molecule detection method, CaT-SMelor, uses a similar principle but requires the TF to be immobilized to microcrystalline cellulose beads and only gives a fluorescent readout (8). Other systems require the use of aptamers, which may limit the range of small molecules for detection (10-12).

Another benefit of this CRISPR based biosensor is the modularity of the readout mechanism. The reporter DNA is modified to be compatible with commercially available lateral flow assays. This provides a way to utilize this biosensor for testing samples outside of a laboratory setting in a relatively easy to use manner.

In addition, Cas12a exhibits a multi-turnover mechanism, meaning one active enzyme cleaves many reporters. This provides built in signal amplification, which is not typically found in protein and small molecule in-vitro detection methods. Methods utilizing nucleic acid-based reactions like transcription provide similar effects. Methods such as ROSALIND have applied this to small molecule detection, but detection limits fall short compared to the Cas12a readout reported here. For example, ROSALIND assays report an LOD of 0.2 μM for doxycycline, where the biosensor assays described herein demonstrate an LOD varying from 80 nM to 900 pM.

The DNA binding site can also be mutated to alter the aTF binding affinity for tunable detection limits (34).

In another aspect, provided herein is a method for using the biosensors described herein for detecting a target molecule or analyte of interest. The method includes contacting the sample with the dsDNA substrate, the DNA binding protein, the gRNA, the Cas enzyme, and the ssDNA reporter construct described herein. Presence of the target analyte in the sample is then detected by signal generated from cleavage of the ssDNA reporter. The methods and sensors can be implemented in multiplexed reactions to enable detection of multiple analytes at the same time. This capability can be exploited to enable panel-based tests for multiple analytes or to improve assay specificity.

In another aspect, provided herein is a kit comprising the biosensors described herein. The kit may comprise, consist of, or consist essentially of a biosensor device comprising a plurality of synthetic dsDNA substrates, each substrate comprising an operator sequence specific to a different binding protein, and a protospacer adjacent motif (PAM) sequence; a plurality of binding proteins, wherein each binding protein binds to one of the operator sequences; a ssDNA reporter construct comprising a detectable tag; a Cas nuclease that exhibits collateral ssDNase activity; and a plurality of gRNAs, wherein each gRNA comprises the reverse complement of one of the operator sequences, and binds to the PAM sequence. The kit may include a buffer. The buffer may be dried with the biosensor reagents, and rehydrated with water. The kit may further include a dropper for dispensing controlled volumes of liquid, such as a controlled volume disposable Pasteur pipette. The kit may further include a written insert component comprising instructions for detecting a target molecule in a sample according to methods of this disclosure.

The term “detect” or “detection” as used herein indicates the determination of the existence, presence or fact of a target or signal in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through visually detectable signals: fluorescence, spectra, or images from a target of interest or a probe attached to the target.

Nucleic acids and/or other moieties of the invention may be isolated. As used herein, “isolated” means to separate from at least some of the components with which it is usually associated whether it is derived from a naturally occurring source or made synthetically, in whole or in part.

Nucleic acids and/or other moieties of the invention may be purified. As used herein, purified means separate from the majority of other compounds or entities. A compound or moiety may be partially purified or substantially purified. Purity may be denoted by a weight by weight measure and may be determined using a variety of analytical techniques such as but not limited to mass spectrometry, HPLC, etc.

Any appropriate sample can be used according to the biosensors and methods provided herein. Appropriate samples include without limitation, food samples, drinking water, environmental samples, and agricultural products. In some embodiments the biosensors and methods provided herein are used in food safety and food biosecurity applications, such as screening food products and materials used in food processing or packaging for the presence of pathogens in biological and/or non-biological samples. In other embodiments, the sample is a biological sample obtained from an individual (e.g., a human subject, a non-human mammal). The sample is, in some cases, a diagnostic sample. The sample type will vary depending on the target pathogen. For example, diagnostic samples can be a serum sample, blood sample, sputum sample, urine sample, or other biological fluid. In some cases, serum samples have been frozen (e.g., at −80° C.) prior to testing. In some cases, samples appropriate for use according to the methods provided herein are “non-biological” in whole or in part. Non-biological samples include, without limitation, plastic and packaging materials, paper, clothing fibers, and metal surfaces.

Other applications for which the methods provided herein include, without limitation, profiling species in an environment (e.g., water); profiling species in an human or animal microbiome; food safety applications (e.g., detecting the presence of a pathogenic species, determining or confirming food source/origin such as type of animal or crop plant); obtaining patient expression profiles (e.g., detecting expression of a gene or panel of genes (e.g., biomarkers) to monitor the patient's response to a therapeutic regimen, to select a therapeutic regimen suitable for the patient, or to detect exposure of the patient to a toxin or environmental agent that affects expression of the gene or a panel of genes.

In some cases, the device is used with a portable electronic reader. In this manner, the electronic reader serves as companion technology that provides robust and quantitative measurements of device outputs. In some embodiments, the electronic reader comprises readily available consumer components, open-source code, and laser-cut acrylic housing, and is powered by a rechargeable lithium ion battery. The electronic reader can further comprise an onboard data storage unit. In some cases, to achieve sensitive detection of toehold switch signal output, an acrylic chip that holds the freeze-dried, paper-based reactions is placed into the reader between a light source (e.g., to read optical density at excitation and emission wavelengths of light appropriate for and characteristic of a particular detectable reporter) and electronic sensors. In some cases, the light source is a light emitting diode (LED) or organic light emitting diode (OLED) light source. Samples can be read using onboard electronics. In this manner, a portable electronic reader can provide low-noise measurements of changes associated with the reporter element including changes in light transmission due to LacZ-mediated color change.

In certain embodiments, provided herein is a device for identifying target analytes, comprising a preserved paper test article, wherein the methods described herein are performed using the preserved paper test article. In some cases, the paper test article is preserved by freeze-drying.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of” “Consisting essentially of” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present invention has been described in terms of one or more preferred embodiments, and it should be appreciated that many equivalents, alternatives, variations, and modifications, aside from those expressly stated, are possible and within the scope of the invention.

### EXAMPLES

The following Examples are illustrative and should not be interpreted to limit the scope of the claimed subject matter. The examples demonstrate a highly modular and sensitive small molecule detection method using CRISPR/Cas12a (cpf1). This provides an intrinsic amplification step that is uncommon to protein and small molecule-based detection platforms but employs a simpler procedure. It also proves to be highly modular considering only the gRNA needs to be reprogrammed for a new small molecule/transcription factor set to interact with Cas12a. This method also displays flexibility by having interchangeable readout mechanisms, both on fluorescence and paper-based platforms. The results show this biosensor can detect 10 different small molecules including antibiotics with tunable detection limits as low as 0.9-4 nM. The biosensor can also function in different water samples and can be implemented in a field deployable assay using lateral flow devices.

### Example 1—Materials and Methods

Construction of dsDNA and gRNA

All operator sequences were turned into Cas12a target sequences by increasing the length to a total of 24 nucleotides using NUPACK online software to generate added bases with minimal unwanted secondary structure. The PAM sequence TTTA was added on the 5′ end of the nontarget strand (TAAA on 3′ of target). An 8-nt clamp was also added on both sides of the complete target to ensure the PAM site remained double stranded by preventing the ends from breathing open. Initial tests showed the clamps were necessary for activation of Cas12a, with little to no signal given from targets without them. The gRNA consists of the standard hairpin sequence for 1baCas12a (UAAUUUCUACUAAGUGUAGAU) (SEQ ID NO: 1) and complementary target sequence starting after the PAM site. All dsDNA substrate and gRNA sequences are provided in Table 1. For guide sequences, the T7 promoter sequence is bolded, the conserved Cas12a hairpin is underlined, and the spacer region is italicized for the first sequence (TetR).

TABLE 1
DNA and RNA sequences used for biosensor

| Biosensor | Target | Docking | Promoter |
|---|---|---|---|
| TetR | CCTACCTAGCCAGTC | GTGTGAGGTTTATCT | TCTCTATCACTGAT |
| | CCTATCAGTGATAGA | CTATCACTGATAGGG | AGGGA |
| | GATAAACCTCACAC | ACTGGCTAGGTAGG | (SEQ ID NO: 4) |
| | (SEQ ID NO: 2) | (SEQ ID NO: 3) | |
| LacI | CCTACCTACCTCGCC | GTGTGAGGTTTATTG | TTGTGAGCGGATAA |
| | TTGTTATCCGCTCAC | TGAGCGGATAACAAG | CAA |
| | AATAAACCTCACAC | GCGAGGTAGGTAGG | (SEQ ID NO: 7) |
| | (SEQ ID NO: 5) | ((SEQ ID NO: 6) | |
| PhiF | CCTACCTAACCTTAA | GTGTGAGGTTTATGA | ATGATACGAAACGT |
| | CGATACGGTACGTTT | TACGAAACGTACCGT | ACCGTATCGTTAAG |
| | CGTATCATAAACCTC | ATCGTTAAGGTTAGG | GT |
| | ACAC | TAGG | (SEQ ID NO: 10) |
| | (SEQ ID NO: 8) | (SEQ ID NO: 9) | |
| CymR | CCTACCTAATAATAC | GTGTGAGGTTTAAAC | AACAAACAGACAAT |
| | AAACAGACCAGATTG | AAACAGACAATCTGG | CTGGTCTGTTTGTA |
| | TCTGTTTGTTTAAAC | TCTGTTTGTATTATT | TTAT |
| | CTCACAC | AGGTAGG | (SEQ ID NO: 13) |
| | (SEQ ID NO: 11) | (SEQ ID NO: 12) | |
| BetL | CCTACCTACCATTGA | GTGTGAGGTTTATTA | ATTGATTGGACGTT |
| | TTGGACGTTCAATAT | TATTGAACGTCCAAT | CAATATAA |
| | AATAAACCTCACAC | CAATGGTAGGTAGG | (SEQ ID NO: 16) |
| | (SEQ ID NO: 14) | (SEQ ID NO: 15) | |
| MphR | CCTACCTAGATTCCA | GTGTGAGGTTTGAAT | AAATGAATATAACC |
| | CCTAAATGTAACAGT | ATAACCGACGTGACT | GACGTGACTGTTAC |
| | CACGTCGGTTATATT | GTTACATTTAGGTGG | ATTTAGGTGG |
| | CAAACCTCACAC | AATCTAGGTAGG | (SEQ ID NO: 19) |
| | (SEQ ID NO: 17) | (SEQ ID NO: 18) | |

| Biosensor | gRNA |
|---|---|
| TetR | GCGCUAAUACGACUCACUAUAGGGUAAUUUCUACUAAGUGUAGAUU |
| | CUCUAUCACUGAUAGGGACUGGC |
| | (SEQ ID NO: 20) |
| LacI | GCGCUAAUACGACUCACUAUAGGGUAAUUUCUACUAAGUGUAGAUU |
| | UGUGAGCGGAUAACAAGGCGAGG |
| | (SEQ ID NO: 21) |
| PhiF | GCGCUAAUACGACUCACUAUAGGGUAAUUUCUACUAAGUGUAGAUU |
| | GAUACGAAACGUACCGUAUCGUU |
| | (SEQ ID NO: 22) |
| CymR | GCGCUAAUACGACUCACUAUAGGGUAAUUUCUACUAAGUGUAGAUA |
| | ACAAACAGACAAUCUGGUCUGUU |
| | (SEQ ID NO: 23) |
| BetL | GCGCUAAUACGACUCACUAUAGGGUAAUUUCUACUAAGUGUAGAUU |
| | UAUAUUGAACGUCCAAUCAAUGG |
| | (SEQ ID NO: 24) |
| MphR | GCGCUAAUACGACUCACUAUAGGGUAAUUUCUACUAAGUGUAGAUA |
| | AUAUAACCGACGUGACUGUUACA |
| | (SEQ ID NO: 25) |
| Transcription | |
| Factor | DNA Sequence |
| TetR | ATGTCCAGATTAGATAAAAGTAAAGTGATTAACAGCGCATTAGAGC |
| | TGCTTAATGAGGTCGGAATCGAAGGTTTAACAACCCGTAAACTCGC |
| | CCAGAAGCTAGGTGTAGAGCAGCCTACATTGTATTGGCATGTAAAA |
| | AATAAGCGGGCTTTGCTCGACGCCTTAGCCATTGAGATGTTAGATA |
| | GGCACCATACTCACTTTTGCCCTTTAGAAGGGGAAAGCTGGCAAGA |
| | TTTTTTACGTAATAACGCTAAAAGTTTTAGATGTGCTTTACTAAGT |
| | CATCGCGATGGAGCAAAAGTACATTTAGGTACACGGCCTACAGAAA |
| | AACAGTATGAAACTCTCGAAAATCAATTAGCCTTTTTATGCCAACA |
| | AGGTTTTTCACTAGAGAATGCATTATATGCACTCAGCGCTGTGGGG |
| | CATTTTACTTTAGGTTGCGTATTGGAAGATCAAGAGCATCAAGTCG |
| | CTAAAGAAGAAAGGGAAACACCTACTACTGATAGTATGCCGCCATT |
| | ATTACGACAAGCTATCGAATTATTTGATCACCAAGGTGCAGAGCCA |
| | GCCTTCTTATTCGGCCTTGAATTGATCATATGCGGATTAGAAAAAC |
| | AACTTAAATGTGAAAGTGGGTCCTAA |
| | (SEQ ID NO: 26) |
| LacI^{AM} | ATGAAACCAGTAACGTTATACGATGTCGCAGAGTATGCCGGTGTCT |
| | CTTATATGACCGTTTCCCGCGTGGTGAACCAGGCCAGCCACGTTTC |
| | TGCGAAAACGCGGGAAAAAGTGGAAGCGGCGATGGTGGAGCTGAAT |
| | TACATTCCCAACCGCGTGGCACAACAACTGGCGGGCAAACAGTCGT |
| | TGCTGATTGGCGTTGCCACCTCCAGTCTGGCCCTGCACGCGCCGTC |
| | GCAAATTGTCGCGGCGATTAAATCTCGCGCCGATCAACTGGGTGCC |
| | AGCGTGGTGGTGTCGATGGTAGAACGAAGCGGCGTCGAAGCCTGTA |
| | AAGCGGCGGTGCACAATCTTCTCGCGCAACGCGTCAGTGGGCTGAT |
| | CATTAACTATCCGCTGGATGACCAGGATGCCATTGCTGTGGAAGCT |
| | GCCTGCACTAATGTTCCGGCGTTATTTCTTGATGTCTCTGACCAGA |
| | CACCCATCAACAGTATTATTTACTCCCATGAGGACGGTACGCGACT |
| | GGGCGTGGAGCATCTGGTCGCATTGGGTCACCAGCAAATCGCGCTG |
| | TTAGCGGGCCCATTAAGTTCTGTCTCGGCGCGTCTGCGTCTGGCTG |
| | GCTGGCATAAATATCTCACTCGCAATCAAATTCAGCCGATAGCGGA |
| | ACGGGAAGGCGACTGGAGTGCCATGTCCGGTTTTCAACAAACCATG |
| | CAAATGCTGAATGAGGGCATCGTTCCCACTGCGATGCTGGTTGCCA |
| | ACGATCAGATGGCGCTGGGCGCAATGCGCGCCATTACCGAGTCCGG |
| | GCTGCGCGTTGGTGCGGATATCTCGGTAGTGGGATACGACGATACC |
| | GAAGATAGCTCATGTTATATCCCGCCGTTAACCACCATCAAACAGG |
| | ATTTTCGCCTGCTGGGGCAAACCAGCGTGGACCGCTTGCTGCAACT |
| | CTCTCAGGGCCAGGCGGTGAAGGGCAATCAGCTGTTGCCAGTCTCA |
| | CTGGTGAAAAGAAAAACCACCCTGGCGCCCAATACGCAAACCGCCT |
| | CTCCCCGCGCGTTGGCCGATTCATTAATGCAGCTGGCACGACAGGT |
| | TTCCCGACTGGAAAGCGGGCAGTGA |
| | (SEQ ID NO: 27) |
| PhIF^{AM} | ATGGCACGTACCCCGAGCCGTAGCAGCATTGGTAGCCTGCGTAGTC |
| | CGCATACCCATAAAGCAATTCTGACCAGCACCATTGAAATCCTGAA |
| | AGAATGTGGTTATAGCGGTCTGAGCATTGAAAGCGTGGCACGTCGC |
| | GCCGGTGCAGGCAAACCGACCATTTATCGTTGGTGGACCAACAAAG |
| | CAGCACTGATTGCCGAAGTGTATGAAAATGAAATCGAACAGGTACG |
| | TAAATTTCCGGATTTGGGTAGCTTTAAAGCCGATCTGGATTTTCTG |
| | CTGCATAATCTGTGGAAAGTTTGGCGTGAAACCATTTGTGGTGAAG |
| | CATTTCGTTGTGTTATTGCAGAAGCACAGTTGGACCCTGTAACCCT |
| | GACCCAACTGAAAGATCAGTTTATGGAACGTCGTCGTGAGATACCG |
| | AAAAAACTGGTTGAAGATGCCATTAGCAATGGTGAACTGCCGAAAG |
| | ATATCAATCGTGAACTGCTGCTGGATATGATTTTTGGTTTTTGTTG |
| | GTATCGCCTGCTGACCGAACAGTTGACCGTTGAACAGGATATTGAA |
| | GAATTTACCTTCCTGCTGATTAATGGTGTTTGTCCGGGTACACAGT |
| | GTTGATAA |
| | (SEQ ID NO: 28) |
| CymR^{AM} | ATGAGCCCGAAACGTCGTACCCAGGCAGAACGTGCAATGGAAACCC |
| | AGGGTAAACTGATTGCAGCAGCACTGGGTGTTCTGCGTGAAAAAGG |
| | TTATGCAGGTTTTCGTATTGCAGATGTTCCGGGTGCAGCCGGTGTT |
| | AGCCGTGGTGCACAGAGCCATCATTTTCCGACCAAACTGGAACTGC |
| | TGCTGGCAACCTTTGAATGGCTGTATGAGCAGATTACCGAACGTAG |
| | CCGTGCACGTCTGGCAAAACTGAAACCGGAAGATGATGTTATTCAG |
| | CAGATGCTGGATGATGCAGCAGAATTTTTTCTGGATGATGATTTTA |
| | GCATCGGCCTGGATCTGATTGTTGCAGCAGATCGTGATCCGGCACT |
| | GCGTGAAGGTATTCAGCGTACCGTTGAACGTAATCGTTTTGTTGTT |
| | GAAGATATGTGGCTGGGTGTGCTGGTGAGCCGTGGTCTGAGCCGTG |
| | ATGATGCCGAAGATATTCTGTGGCTGATTTTTAACAGCGTTCGTGG |
| | TCTGGTAGTTCGTAGCCTGTGGCAGAAAGATAAAGAACGTTTTGAA |
| | CGTGTGCGTAATAGCACCCTGGAAATTGCACGTGAACGTTATGCAA |
| | AATTCAAACGTTGA |
| | (SEQ ID NO: 29) |
| BetL^{AM} | ATGCCGAAACTGGGTATGCAGAGCATTCGTCGTCGTCAGCTGATTG |
| | ATGCAACCCTGGAAGCAATTAATGAAGTTGGTATGCATGATGCAAC |
| | CATTGCACAGATTGCACGTCGTGCCGGTGTTAGCACCGGTATTATT |
| | AGCCATTATTTCCGCGATAAAAACGGTCTACTGGAAGCAACCATGC |
| | GTGATATTACCAGCCAGCTGCGTGATGCAGTTCTGAATCGTCTGCA |
| | TGCACTGCCGCAGGGTAGCGCAGAACAGCGTCTGCAGGCAATTGTT |
| | GGTGGTAATTTTGATGAAACCCAGGTTAGCAGCGCAGCAATGAAAG |
| | CATGGCTGGCATTTTGGGCAATCAGCATGCATCAGCCGATGCTGTA |
| | TCGTCTGCAGCAGGTTAGCAGTCGTCGTCTGCTGAGCAATCTGGTT |
| | AGCGAATTTCGTCGTGAACTGCCTCGTGAACAGGCACAAGAGGCAG |
| | GTTATGGTCTGGCAGCACTGATTGATGGTCTGTGGCTGCGTGCAGC |
| | ACTGAGCGGTAAACCGCTGGATAAAACCCGTGCAAATAGCCTGACC |
| | CGTCATTTTATCACCCAGCATCTGCCGACCGATTGA |
| | (SEQ ID NO: 30) |
| MphR | tataccatgccgcgtccgaaattaaaatcggatgacgaagttcttg |
| | aggcagcgactgtagtattgaaacgctgtggtcccattgagtttac |
| | gctttctggggtcgcgaaagaagttggtcttagccgcgcagcgttg |
| | atccaacgttttaccaaccgcgacacgctgttggtgcgtatgatgg |
| | aacgtggcgtggagcaggtgcgtcactacttaaatgcgatccccat |
| | tggggcgggccctcagggtttatgggagtttcttcaagttttagtg |
| | cgtagcatgaatacgcgtaatgacttctctgtaaactacttgattt |
| | cgtggtacgagcttcaagtccccgaattgcgcacactggcgattca |
| | acgcaaccgcgcagtagtagaaggtatccgcaagcgtttgcctcct |
| | ggggcccctgcggcggctgagttgctgctgcatagcgtgattgcgg |
| | gtgccacgatgcagtgggcggtggacccggacggtgaattagccga |
| | tcatgtattagcccagatcgctgcgatcttgtgtttaatgttcccc |
| | gaacatgatgactttca |
| | (SEQ ID NO: 31) |

All DNAs used in the study were synthesized from Integrated DNA Technologies. Actual gRNAs were constructed using assembly PCR primers containing a T7 promoter sequence, Cas12a conserved hairpin, and a complementary target sequence. Ampliscribe T7-Flash Transcription Kits (ASF3257) were used to transcribe PCR products and RNA was purified using a Monarch RNA Cleanup Kit (NEB T2040L).

### Plasmid Construction

Genes for all proteins except for MphR were taken from plasmids from the Marionette system (7) and inserted into pET15b plasmids for expression. The MphR protein came from plasmid pJBL715 from Julius Lucks (Addgene plasmid #140385; http://n2t.net/addgene:140385; RRID:Addgene 140385) (3).

### Protein Expression and Purification

Overexpression and purification of TetR/pET15b and Lad/pET15b plasmids containing TetR and Lad genes under T7 promoter regulation were transformed into BL21 Star (DE3) cells. Single colonies of transformed cells were used to grow overnight culture at 37° C. in lysogeny broth supplemented with carbenicillin (100 μg/ml). Large expression cultures were inoculated with overnight cultures at 1:100 dilution and were shaken at 37° C. These cultures were induced with 0.5 mM IPTG at OD600 and further grown for 4 h at 37° C. Cultures were then centrifuged at 3000 g for 30 min to collect the cell pellet and stored at −80° C. Later, the cell pellet was resuspended in lysis buffer (10 mM Tris-HC1, pH 8.0, 500 mM NaCl, 1 mM TCEP, 20 mM Imidazole, cOmplete™ Mini EDTA-free Protease Inhibitor Cocktail) and sonicated on ice for 5 min (1 sec on, 2 sec off, 25% amplitude) to break open the cells and release the transcription factor protein. Cell lysate was centrifuged at 30,000 g for 30 mins and supernatant was subjected to Ni-NTA gravity column to purify the his-tagged proteins. The eluted protein was dialyzed using SnakeSkin™ Dialysis Tubing and stored in Tris buffer (10 mM Tris-HCl, pH 8.0, 500 mM NaCl) at 4° C. for 1 week or in 50% glycerol at −20° C. Sequences encoding for the transcription factors are provided in Table 1.

### Detection and Cas12a Cleavage Reactions

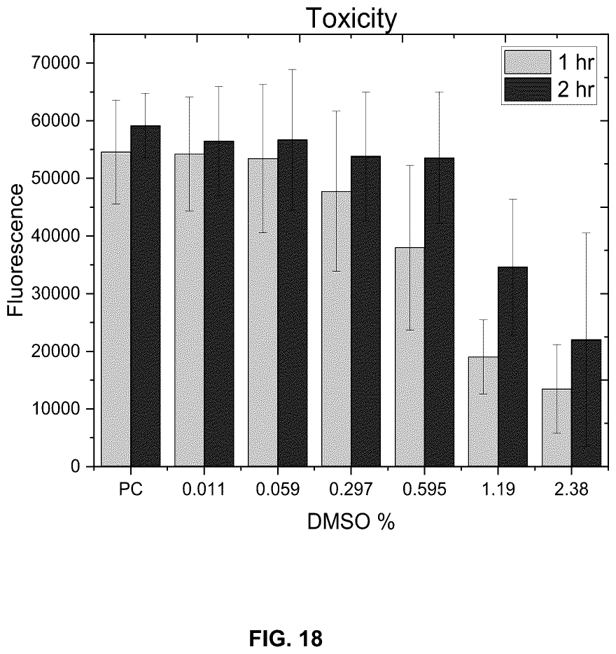

Target and nontarget strands (NTS) were annealed at a 1:2 ratio in 1× TAE+12.5 mM Mg²⁺ for 5 minutes at 95° C., then snap cooled. To form the DNA/TF complex, 100 nM dsTarget complexes were incubated with 10 μM corresponding transcription factors (unless otherwise stated) for 15 minutes at 37° C. in incubation buffer (40 mM Tris-HCl 8 mM MgCl₂, 20 mM NaCl, pH 8). Small molecules were added next, following the same incubation procedure. Prior to addition, small molecules were suspended in water, DMSO or ethanol. FIG. 18 demonstrates that the effects of DMSO on Cas12a cleavage were not detrimental at experimental levels (0.03%). A noticeable impact on cleavage was noticed at 1% DMSO and above.

To monitor small molecule detection, 2 μl of the DNA/TF or DNA/TF/SM mixture were added to 10 μl total Cas12a cleavage reaction. This consisted of: 53 nM EnGen Lba Cas12a (NEB M0653T), 100 nM crRNA, 200 nM 5′-6-FAM-TTTTTTTTTT-BHQ1-3′ (IDT) (SEQ ID NO: 38), and 1× NEBuffer r2.1. Cleavage reactions were then immediately added to a microplate such as a Corning 384-well Black Flat Bottom Plates and read on a SynergyNeo2 Biotek fluorescence plate reader. Fluorescence measurements were taken with 487 nm excitation, 528 nm emission from the bottom of the plate for 2 hours. Fluorescence curves show raw fluorescence images unless otherwise stated. A summary of experimental parameters are shown in Tables 2 and 3.

TABLE 2
Experimental Information: Solubilizing small molecules.

| | | Stock Con- | | |
|---|---|---|---|---|
| Small molecule | Solvent | centration | | Purchase Info |
| Anhydrotetracycline | DMSO | 22 | mM | 37919-100MG-R |
| Doxycycline | Ultra pure H₂O | 100 | mM | D3447-500MG |
| Isopropyl β-D- | Ultra pure H₂O | 100 | mM | I6758-1G |
| 1thiogalactopyranoside | | | | |
| 2,4- | DMSO | 100 | mM | SC-206518 |
| Diacetylphloroglucinol | | | | |
| (DAPG) | | | | |
| Cuminic acid | DMSO | 100 | mM | 268402 |
| Choline Chloride | Ultra pure H₂O | 100 | mM | C7017 |
| Erythromycin | EtOH | 50 | mM | E5389-1G |
| Clarithromycin | EtOH | 1 | mM | PHR1038-500MG |
| Roxithromycin | EtOH | 20 | mM | R4393-1G |
| Azithromycin | EtOH | 10 | mM | PHR-1088-1G |

TABLE 3
Experimental Information: Experimental concentrations
for each TF-SM biosensor.

| TF-SM | DNA nM | TF μM | SM μM |
|---|---|---|---|
| TetR-aTc | 5-20 | 0.5-20 | 0.004-40 |
| TetR-Doxy | 5-20 | 0.5-20 | 0.004-40 |
| PhiF-DAPG | 20 | 2.5 | 0.004-100 |
| CymR-Cuminic acid | 20 | 1 | 0.04-400 |
| LacI-IPTG | 20 | 1-20 | 0.04-500 |
| MphR-Clar | 20 | 1 | 10 |
| MphR-Ery | 5-20 | 1 | 0.0004-40 |
| MphR-Azy | 20 | 1 | 20 |
| MphR-Roxi | 20 | 1 | 500 |
| BetL-Cho | 20 | 2 | 100-250,000 |

### Dose Response Fittings

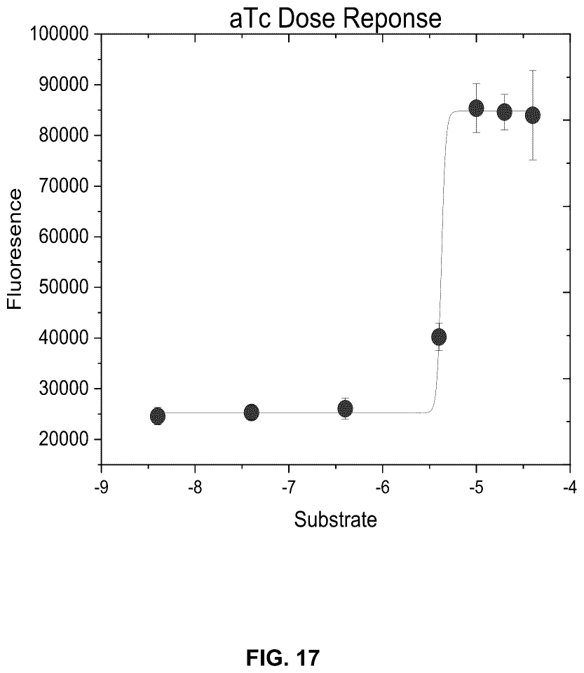

Dose response curves were fit on OriginLab graphing software using equation 1. A1 is the minimum asymptote, A2 is the maximum asymptote, X is the log of a given dosage, and p is the hill slope. The center of the curve is equal to Log x0, so the half response or EC 50=10^{Logx0}. Calculated values for the dose response curve and an example response curve is shown at FIG. 17.

$$y = A 1 + \frac{A 2 - A 1}{1 + 1 {0}^{( {Log}_{x} 0 - x ) p}} \qquad ( 1 )$$

### Lateral Flow

DNA/TF/small molecule samples were incubated and added to Cas12a reactions containing 106 nM Cas12a, 100 nM gRNA, and 200 nM FAM-TTTTTTTTTT-Biotin (SEQ ID NO: 38) or 5′FAM-TTTT(internal T-Biotin)TTTTT-BHQ1 (SEQ ID NO: 38) reporter (IDT). The Cas12a concentration was increased to speed up the reaction time. Samples were incubated for 30 minutes at 37° C. on a thermocycler block. The 10 μl samples were added directly to the dipstick, HybriDetect Universal Lateral Flow Assays from Milenia Biotec (MGHD1) and placed in 100 μl of LF running buffer containing 50% PEG 800. PEG was used to slow capillary flow down, giving reporter strands more time to interact with gold nanoparticle-antibody conjugates before flowing down the assay. This provided cleaner negative results. After 2 minutes, dipsticks were removed and imaged using a cellphone camera.

### Statistical Analysis

All samples were done in replicates of 4. Welch's t-test was used to determine statistical differences in SM+/−samples. (P-value<0.001 ***, P<0.01**).

### Example 2—Design and validation of Cas12a-based biosensors

To implement a small-molecule detection system harnessing Cas12a, a double-stranded DNA (dsDNA) substrate capable of binding to either a transcription factor of interest or Cas12a complexed with gRNA was established (FIG. 1, part a). The dsDNA substrate thus consisted of the TTTV PAM sequence recognized by Cas12a from Lachnospiraceae bacterium adjacent to the DNA operator site of the transcription factor. Binding of Cas12a to the dsDNA was programmed using a gRNA with a spacer overlapping with the TF operator site positioned 20-24 bases adjacent to the PAM site. Flanking base pairs were added to the dsDNA before the PAM site and after the operator sequence to accommodate Cas12a binding and ensure a fully double-stranded PAM site. Incubation of excess transcription factor in the presence of dsDNA substrate enabled the two species to form a complex. The off state of the biosensor was maintained by continued transcription factor binding to the dsDNA substrate, which prevented Cas12a dsDNA substrate interactions, as shown in FIG. 1, part b. In the presence of a reciprocal small molecule, the TF released the dsDNA, revealing the PAM site and dsDNA substrate. Binding of Cas12a/gRNA to the substrate activated Cas12a ssDNA collateral cleavage activity (FIG. 1, part c). Collateral cleavage was then trained on a fluorophore-quencher ssDNA reporter and was detected using a fluorescence plate reader or visualized using lateral flow strips (Error! Reference source not found. 1, part d).

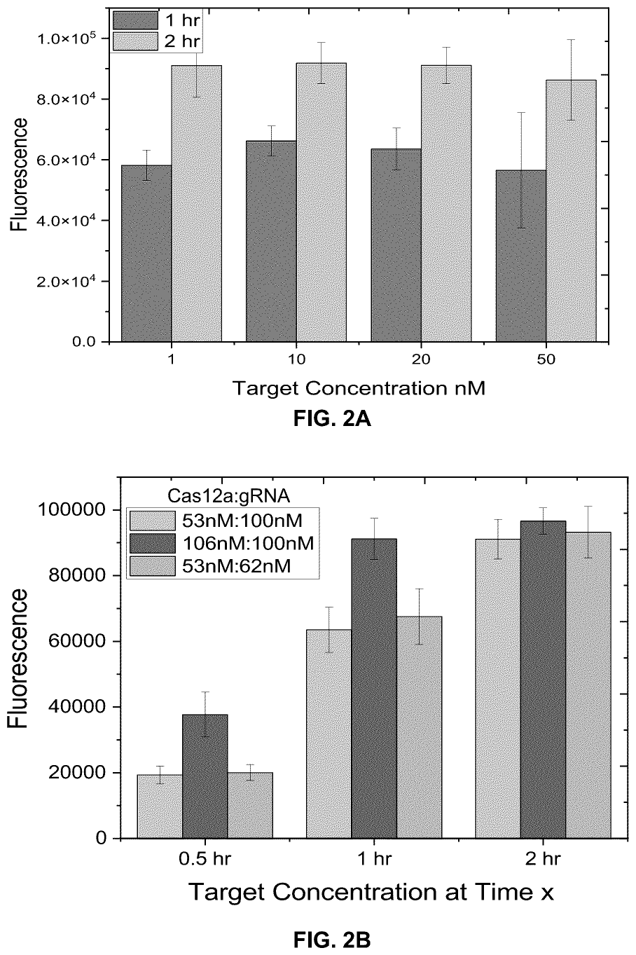

To ensure the transcription factor binds to the DNA substrate with high affinity, the well characterized tetracycline repressor (TetR) and its operator, TetO was used for initial designs. TetR has a high affinity for the 19-bp operator TetO with a K_{d} of 0.18 nM in vitro (27). It also binds to small molecule tetracyclines and the widely used antibiotic, doxycycline, with high affinities. The ability of the dsDNA substrate to activate Cas12a/gRNA complex was first tested by incubating the dsDNA with the Cas12a/gRNA complex in a cleavage reaction mixture containing a fluorophore-quencher ssDNA reporter. Control experiments showed the TetR-specific dsDNA target could activate Cas12a at concentrations ranging from 1 to 50 nM of dsDNA generating similar fluorescence signals (FIG. 2A). An increase in signal was seen between the 1-h and 2-h time points, indicating the reaction had not reached completion at 1 h. The impacts of Cas12a and gRNA on cleavage were monitored by testing different concentrations/ratios of Cas12a to gRNA and monitoring cleavage at 30 min, 60 min, and 120 min (FIG. 2B). Results show that having a higher concentration of Cas12a and gRNA made the reaction go faster for the first hour. After 2 hours, all samples reached near saturation. A slight increase in cleavage was seen with a 106/100 nM ratio of Cas12a/gRNA for the first 60 minutes only, so a concentration of 53 nM/100 nM Cas12a/gRNA was used for all fluorescence studies to conserve enzyme supplies. For lateral flow assays, 106 nM Cas12a and 100 nM gRNA were used.

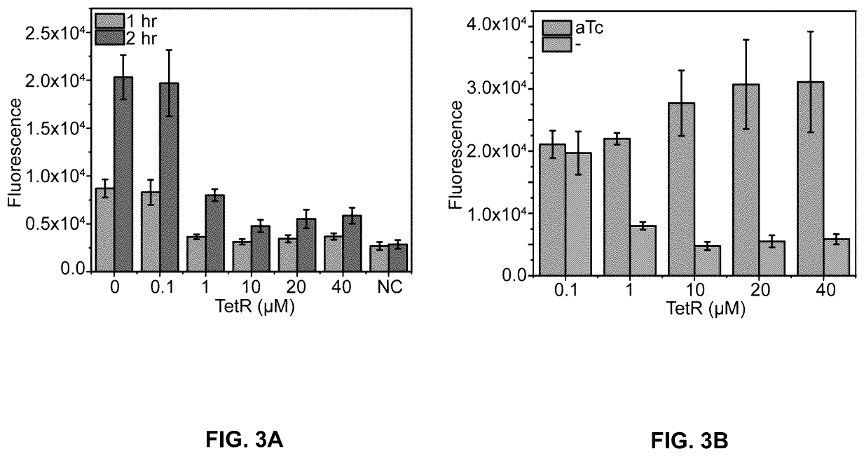

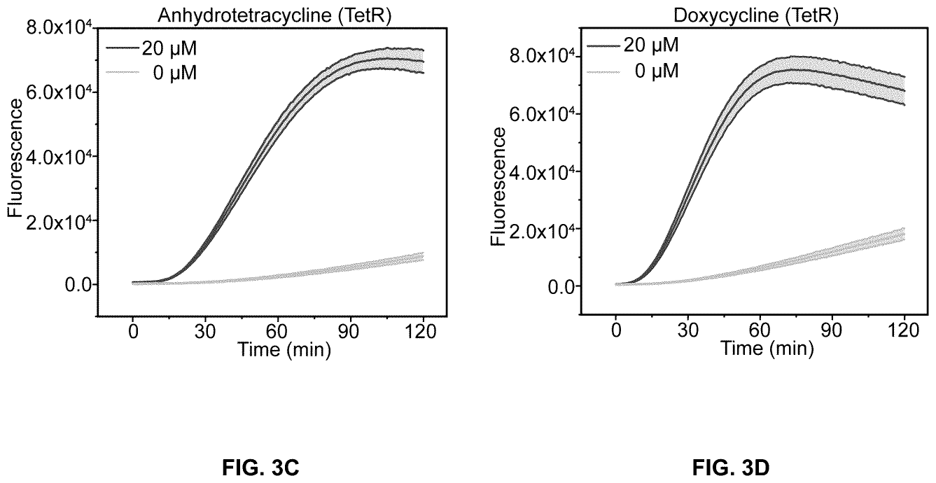

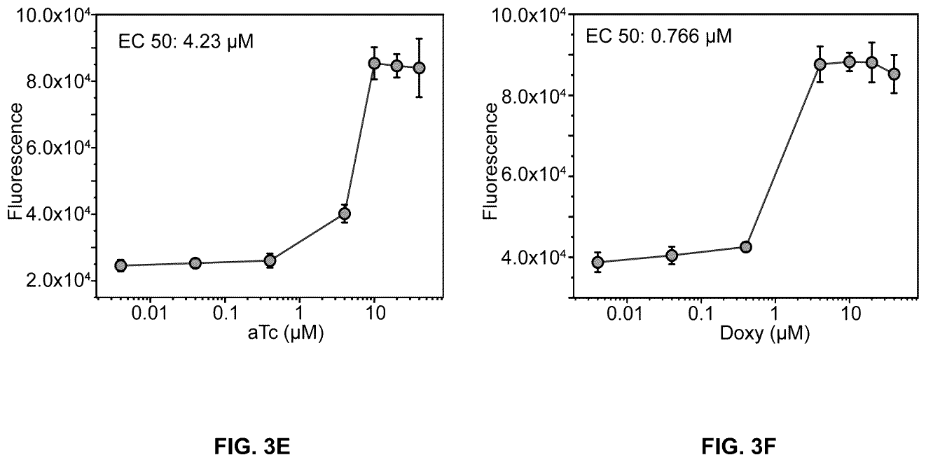

Next, transcription factor mediated repression of Cas12a activation was tested. Purified recombinant TetR was added to the dsDNA substrate and incubated for 15 minutes at 37° C. It was then combined with the Cas12a/gRNA complex and cleavage of ssDNA was monitored. The reactions showed Cas12a suppression compared to the positive control of dsDNA only (PC) with as low as 1 μM TetR (FIG. 3A). Optimum repression was achieved with 10 μM TetR, corresponding to a 50-fold higher dimeric TetR concentration compared to the dsDNA substrate. Time points taken at 1 h and 2 h also showed that repression was well maintained for 10 μM and higher concentrations of TetR for the entire experiment, with fluorescent signals much closer to the negative control (NC) throughout.

To reactivate the sensor, 20 μM anhydrotetracycline-HCl (aTc) or doxycycline (Doxy) was added to the TetR/dsDNA complex and incubated for 15 minutes at 37° C., giving the system time to reach a binding equilibrium before introducing the Cas12a cleavage mix. FIGS. 3C and D show fluorescence kinetic curves over a 2-h timespan for cleavage reactions containing either of the small molecules along with a negative control containing no small molecule. As expected, the plots indicate a high signal increase in small molecule positive samples, where the dsDNA substrate was released from transcription factors. Minimal leakage signal was observed from samples lacking the small molecules where the TF remained bound to the dsDNA.

Transcription factor concentration is not only vital in ensuring that Cas12a access to the dsDNA is hindered, but also in guaranteeing enough TF is occupied by small molecule binding to result in Cas12a activation via free dsDNA substrate. If the TF concentration is too high, there may be enough to occupy both the small molecule and dsDNA in the system, preventing a measurable Cas12a output. The effects of TetR concentration on aTc responsiveness were tested by introducing different concentrations of TF with a fixed aTc concentration. FIG. 3B shows the best on/off ratios between the aTc positive and negative samples using 10 μM and higher concentrations of TetR with 20 μM aTc at 2 h. The results indicate that a 50-fold excess of the TetR dimer is sufficient not only for suppression, but for small molecule responsiveness.

The limit of detection for the biosensor was tested by introducing a range of small molecules from 4 nM to 40 μM using a 50:1 TetR dimer/dsDNA ratio (FIGS. 3E and F). The TetR sensor exhibited a higher sensitivity to Doxy, giving a near maximum fluorescence output with 4 μM Doxy. aTc was slightly less sensitive showing maximum signal at 10 μM a decrease in signal at 4 μM, and complete loss at 0.4 μM. The dose response curve of the biosensor was further assessed using the EC 50 value, which represents the concentration of a drug or analyte needed to reach a half maximum output signal based on a protein-analyte interaction. The EC 50 values were determined to be 766 nM for Doxy and 4.23 μM for aTc. These results show that TetR binds to the dsDNA with high enough affinity to block Cas12a interactions but can be displaced from the dsDNA when the cognate small molecule is introduced, enabling activation of Cas12a.

### Example 3—Expanding Small Molecule Detection

A crucial feature for a biosensor platform is the ability to specifically detect a variety of different molecules. Thus, multiple previously reported transcription factor/small molecule pairs were examined to determine if the biosensor strategy could be applied to an array of different small-molecule analytes. New biosensor sets were designed by incorporating the operators for the TFs LacL MphR, CymR, PhiF, and BetL into double-stranded substrates. However, MphR, CymR, and PhiF did not provide sufficient binding affinity to the cognate operator to block Cas12a binding. The dissociation constant for Lba Cas12-gRNA and its dsDNA target have been determined to range from 0.13-3.9 nM depending on the presence of DTT reducing agent (28). Conflicting studies have found the K_{d} value can be as low as 54 fM, but this required over 32 hours of incubation with the DNA target (29). When using the TetR system, the binding affinity for TetR to the DNA was high enough to avoid Cas12a competition. When considering some of the other transcription factors, this was not the case. For example, the K_{d} for MphR to its DNA operator is 574 nM (30).

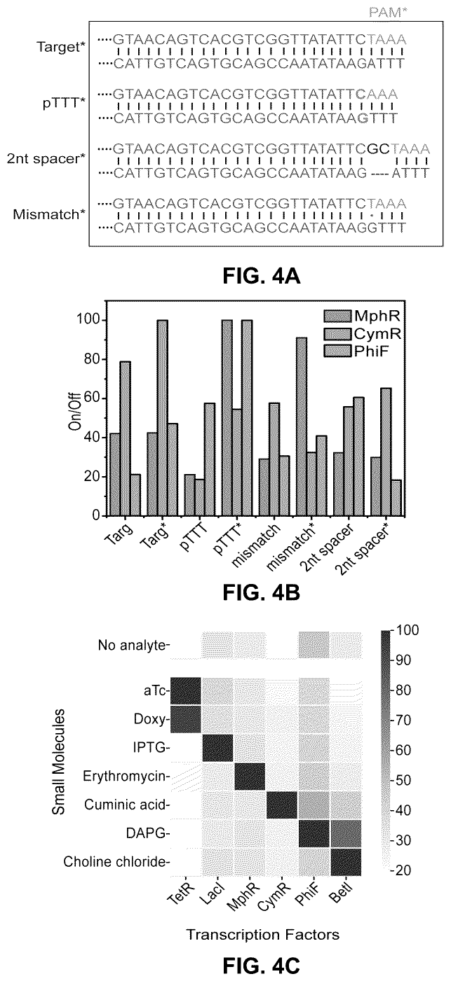

Thus, different dsDNA configurations were examined to establish conditions that favored TF binding over Cas12a/gRNA binding for many TF/dsDNA pairs. Four designs were tested. Design names are shown in bold and the descriptions are as follows: (FIG. 4A) Target, which is the original design; pTTT, where the fourth nucleotide of the PAM sequence is incorporated into the protein operator, meaning it may also be involved in transcription factor binding; mismatch, where the fourth nucleotide of the PAM sequence was mutated, causing a 1 nucleotide bulge in the double stranded substrate; and 2 nt. spacer, where two nucleotides were added between the PAM sequence and operator. All the designs were tested in both sense and antisense orientation of the operator which may impact steric hinderance. Antisense is denoted with “*”.

On and off ratios were calculated for each sample by dividing the target only sample by the target+TF sample, and the data was normalized to the maximum output for each set. The results are plotted in FIG. 4B. FIG. 4C illustrates orthogonality data as fluorescence endpoint values from 1 hour of cleavage when exposed to each small molecule using all transcription factors. Data was normalized by setting the correct target SM to 100. The results show different modifications had a major impact in activation and repression of Cas12a, indicating subtle changes to the DNA could reduce Cas12a/gRNA/DNA affinity. This also demonstrates that slight modifications were enough to favor TF/DNA binding. For both the MphR and PhiF sets, antisense designs that hid the first nucleotide of the PAM sequence worked best. Because the PAM site is recognized by Cas12a, hiding the final nucleotide may reduce its affinity or ability to recognize the dsDNA substrate until the TF is displaced. For the CymR set, the original design in antisense orientation performed best.

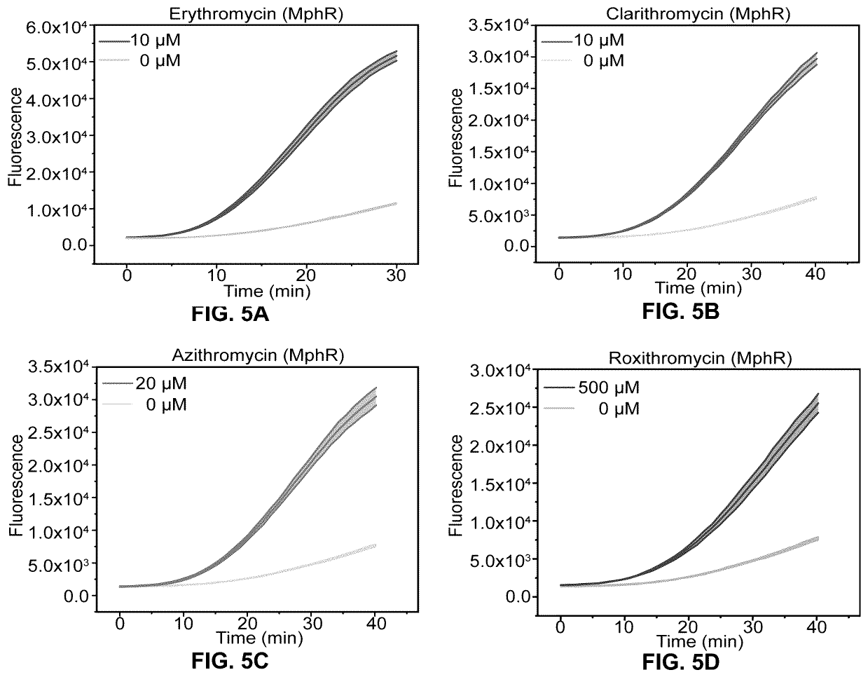

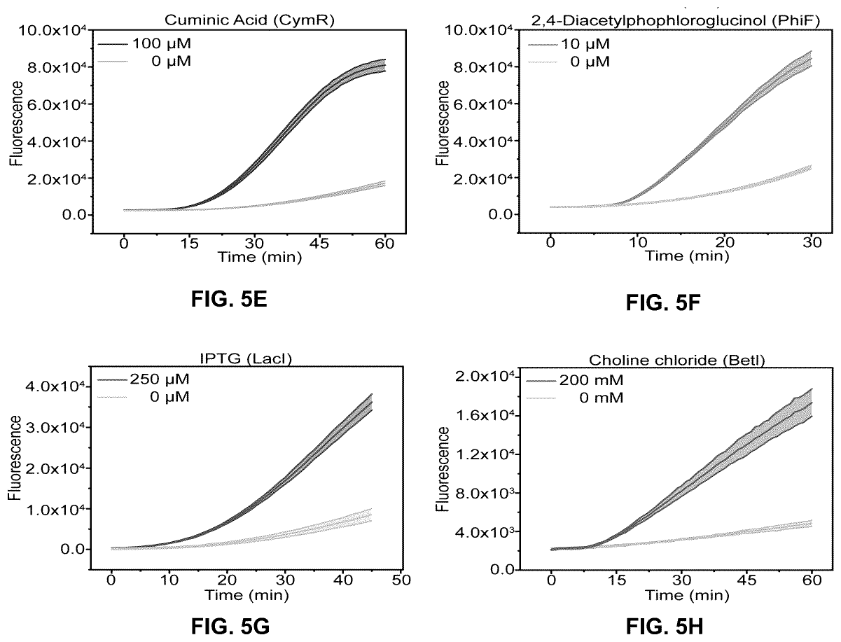

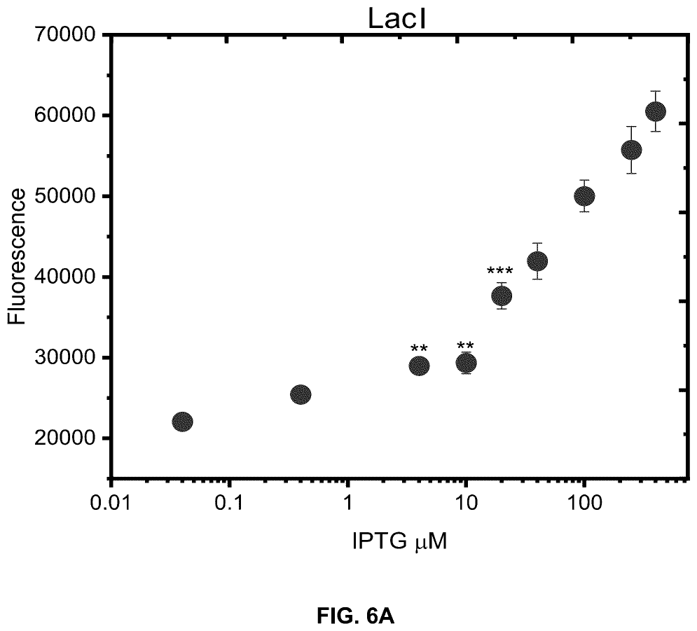

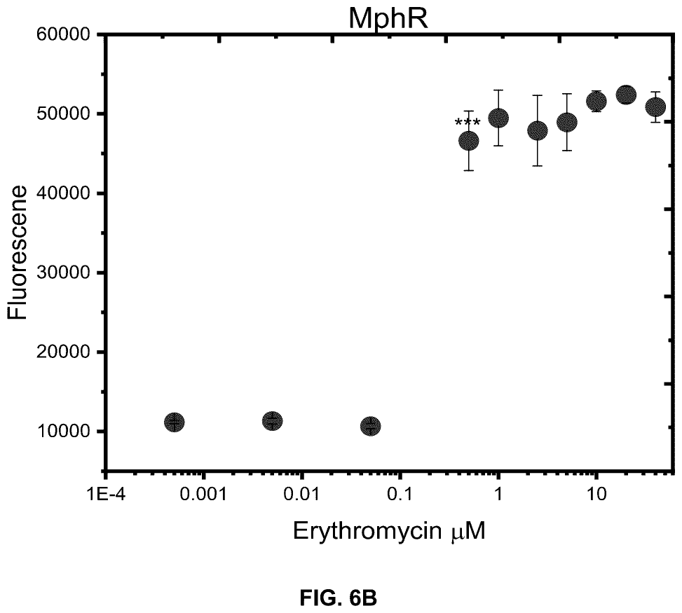

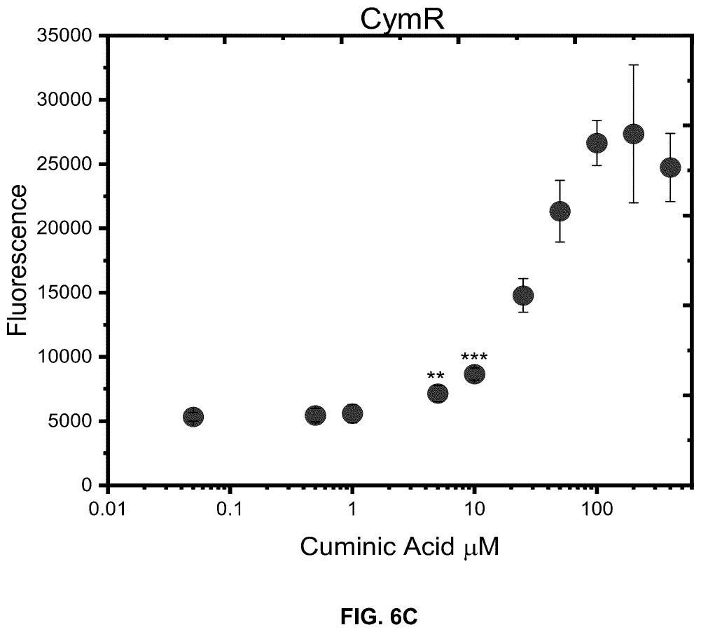

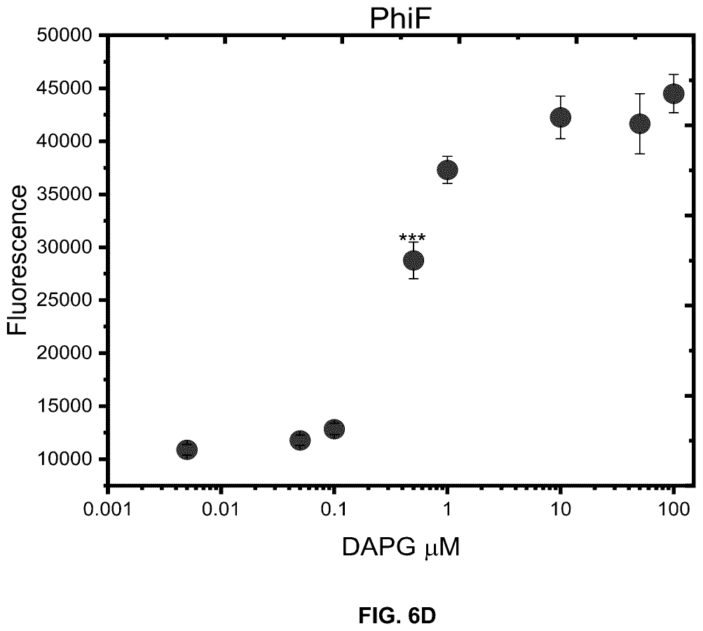

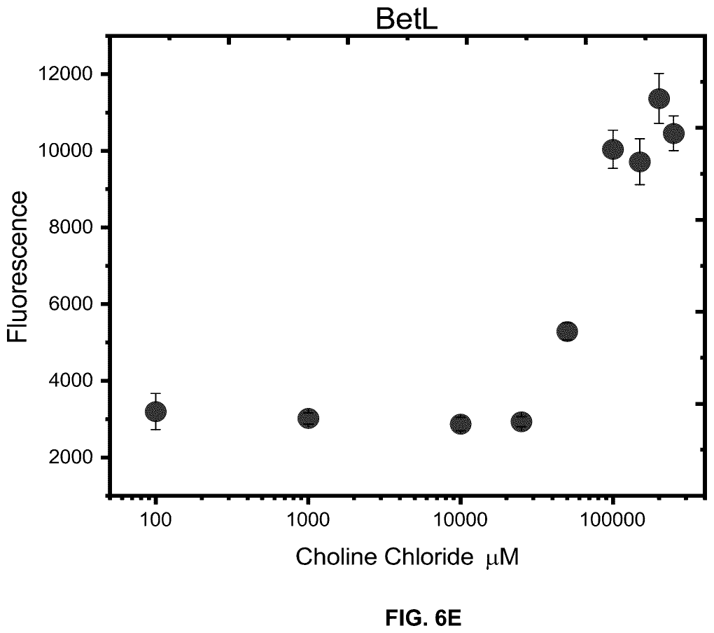

Similar experimental protocols with the Cas12a cleavage reaction were followed using the corresponding recombinant transcription factors, the cognate small molecules, and optimized dsDNA substrates. Fluorescence was measured for each TF with and without the corresponding small molecule analyte over a 30- to 60-minute period (FIG. 5). There was a clear distinction between the positive and negative samples for each TF set, with positive signals typically distinguishable within the first 15 minutes. Detection limits for the different biosensors varied depending on the TF and small molecule affinities. Dose response curves for Lad, MphR, CymR, PhiF, and Betl are shown in FIG. 6. For these experiments, and for the plots shown in FIG. 5, the protein concentrations were optimized for each set depending on the amount of TF required to suppress Cas12a signal. Dose response fittings were performed on the small molecule titration curves and the EC 50 values were calculated for each (FIG. 6). The amount of TF used, and EC 50 values are shown in Table 4. A dose response fitting could not be conducted for Lad because the fluorescence signal never reached saturation.

TABLE 4
Dose Response Parameters and Values

| | | |
|---|---|---|
| Transcription Factor | TF exp. Conc. | EC 50 μM |

| | | | |
|---|---|---|---|
| LacI | 1 | μM | N/A |
| MphR | 1 | μM | 0.34 |
| CymR | 1 | μM | 28.9 |
| PhiF | 2.5 | μM | 0.45 |
| BetL | 2 | μM | 60 mM |

Orthogonality between the analytes and non-cognate TFs was tested by introducing each of the small molecules to each transcription factor with the corresponding dsDNA in the Cas12a cleavage reaction. The results are shown in FIG. 4C with fluorescence intensity normalized to the signal obtained from the correct small molecule. BetI and PhiF showed some crosstalk. Yet for all six TFs, the strongest signals were observed only when the cognate small molecule was introduced, indicating each protein was correctly displaced by its corresponding small molecule.

### Example 4—Tuning Detection Using Parameter Manipulation

The lower end of the detection space for the biosensors described above spanned 100-450 nM for the best working TF/SM pairs. This sensitivity is comparable to other small molecule biosensors that report an overall detection limit in the hundreds of nanomolar range (2, 10, 11). These limits also fit in within relevant concentrations of antibiotics found in ground water sources, which span from 10-50 ug/L and up to 450 ug/L depending on the country. A middle range of 50 ug/L is the equivalent to 111 nM Doxycycline. Although some TF/SM sets were within this range, they were at the higher limit. To extend the utility of the biosensing platform, ways to attain improved detection tunability, lower detection limits, and faster cleavage time were investigated.

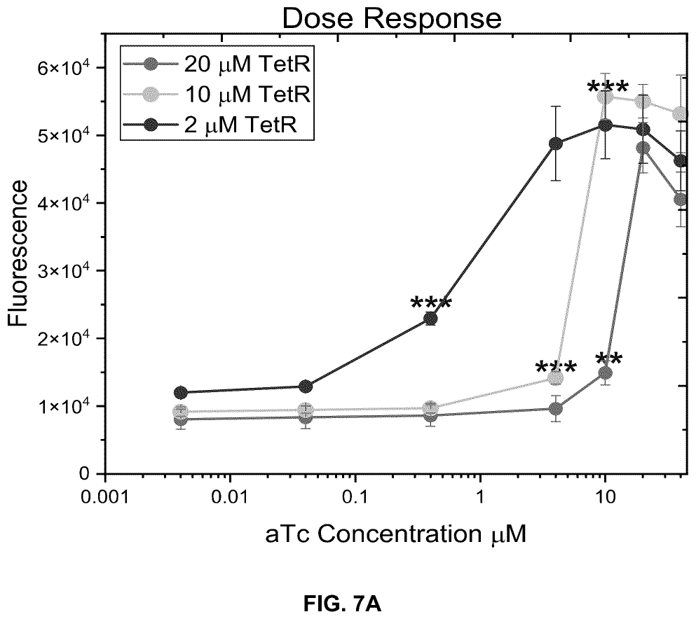

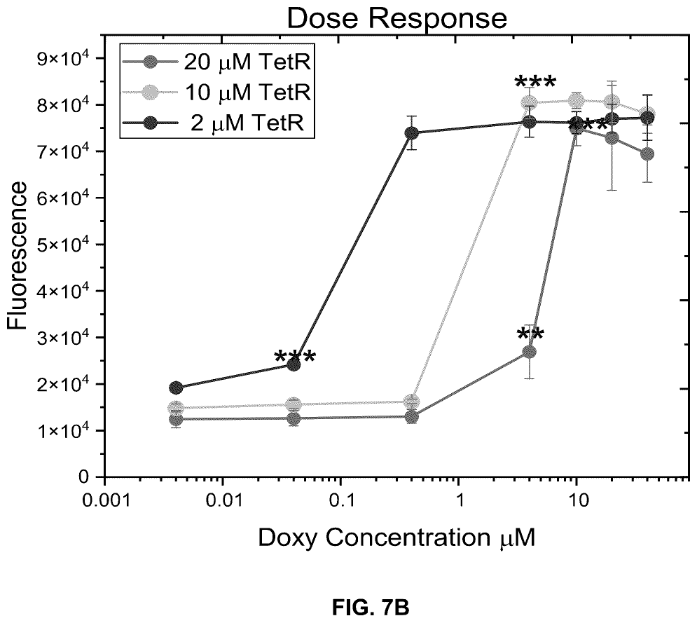

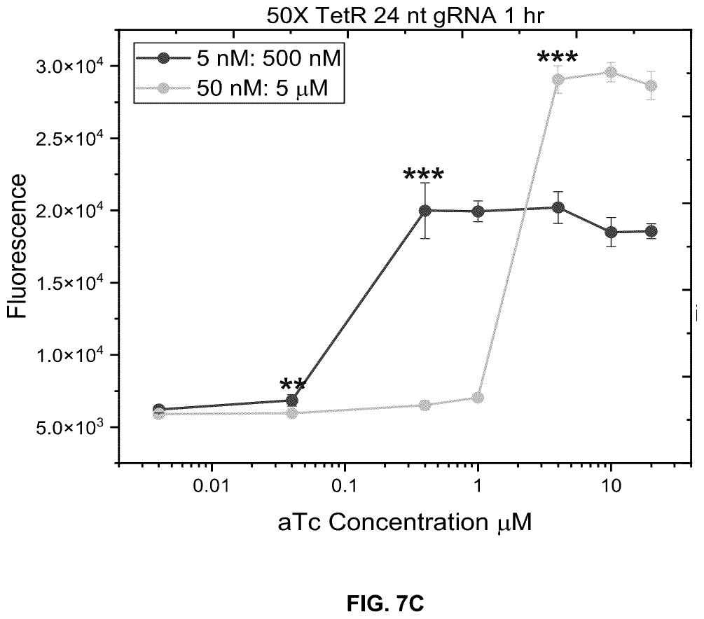

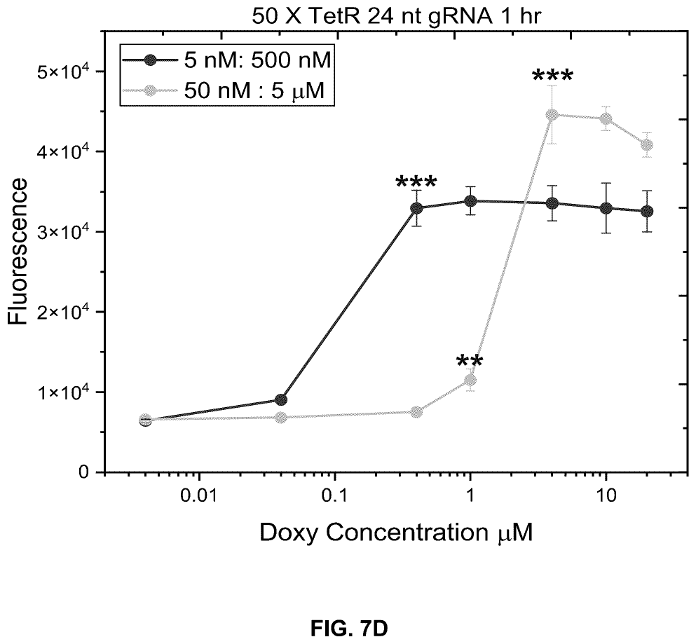

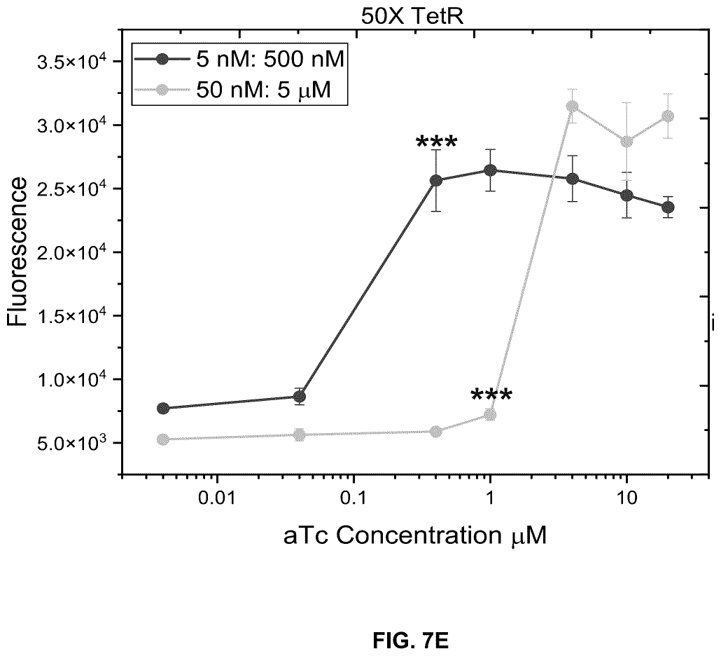

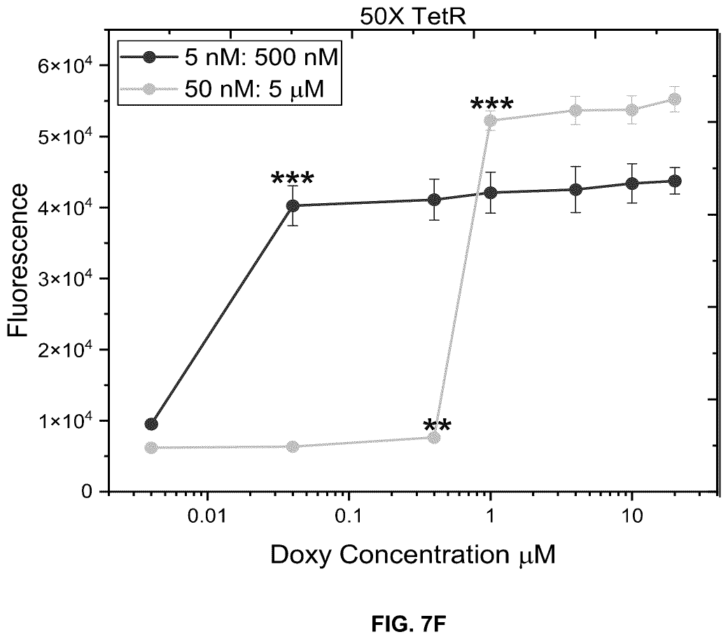

First, the number of small-molecule binding sites in the system was adjusted by varying the TF concentration. As the number of sites decreased, lower concentrations of small molecule was required to achieve the same Cas12a cleavage signal. This was demonstrated by looking at varying doses of aTc and Doxy at three different concentrations of TetR. FIG. 7A displays the dose response curves for aTc and Doxy at 1 h. Similar results were shown at 2 h. A shift in the curves is observed as the TF concentration is reduced, indicating sensitivity is enhanced. Changes in detection were also quantified by considering the EC 50, where the concentration required to achieve half maximum signal for aTc ranged from 10.57 μM to 0.84 μM by lowering the TF concentration (Table 5). The Doxy system saw an EC 50 range from 4 μM to as low as 0.11 μM (Table 6). This demonstrates that by altering the TF concentration, the detection limits can be tuned, providing a level of control for biosensor application.

TABLE 5
aTc Tunability for Speed and Detection Limit

| dsDNA concentration | TF Concentration | | EC:50 aTc (μM) | |
|---|---|---|---|---|
| 100 nM | 20 | μM | 10.57 | |
| | 10 | μM | 4.42 | |
| | 2 | μM | 0.84 | |
| | | | 24 nt gRNA | 20 nt gRNA |
| 50 nM | 5 | μM | 1.77 | 1.51 |
| 5 nM | 500 | nM | 0.057 | 0.051 |

TABLE 6
Doxy Tunability for Speed and Detection Limit

| dsDNA concentration | TF Concentration | | EC:50 Doxy (μM) | |
|---|---|---|---|---|
| 100 nM | 20 | μM | 4.22 | |
| | 10 | μM | 0.95 | |
| | 2 | μM | 0.11 | |
| | | | 24 nt gRNA | 20 nt gRNA |
| 50 nM | 5 | μM | 1.59 | 0.43 |
| 5 nM | 500 | nM | 0.088 | 0.0009 |

Because a 50-fold tetR dimer:DNA ratio provided the lowest leakage in previous sections, a series of reactions maintaining the same ratio at different concentrations were tested with the aim to reduce the number of analyte binding sites while preserving a robust off state. FIG. 7B shows dose-response curves for both aTc and Doxy with a DNA target concentration of 5 nM and 50 nM at 1 h of cleavage. Dropping the DNA down to 5 nM with 500 nM TF resulted in a noticeable shift in detection limit, with the EC 50 for aTc going from 4.42 to 0.05 μM and 0.95 to 0.087 μM for Doxy. Significant changes were not observed in the case of 50 nM target, 1 μM TF. By lowering the overall amount of TetR:DNA in the system, the number of small molecule binding sites was further reduced while maintaining the optimal ratio for DNA-TF repression. Note that all EC 50 values corresponding to experiments consisting of the 50-fold TetR dimer: DNA ratio are bolded in Table 5 and Table 6.

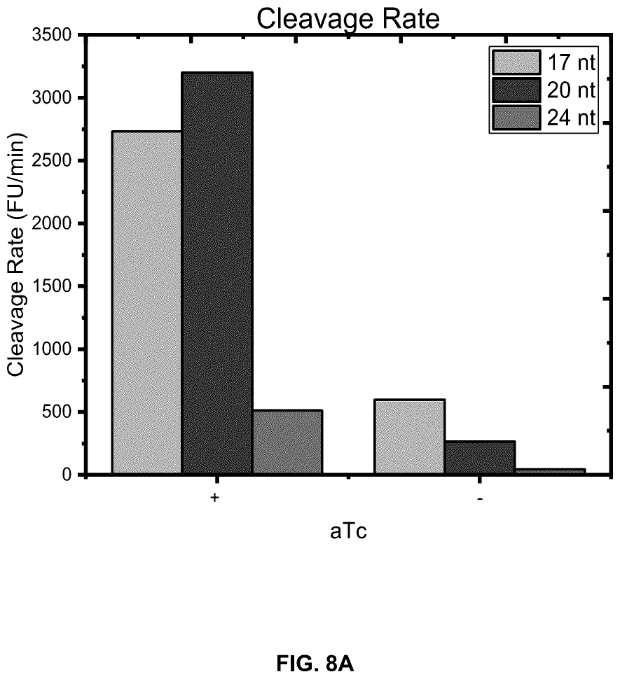

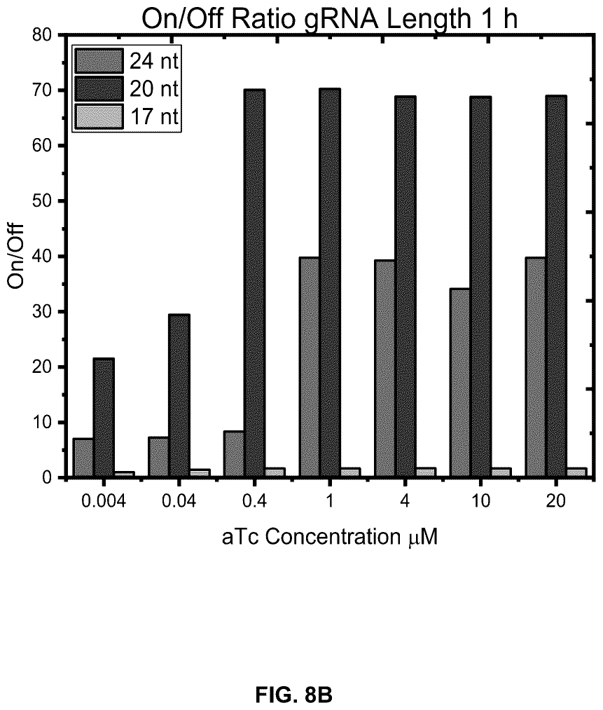

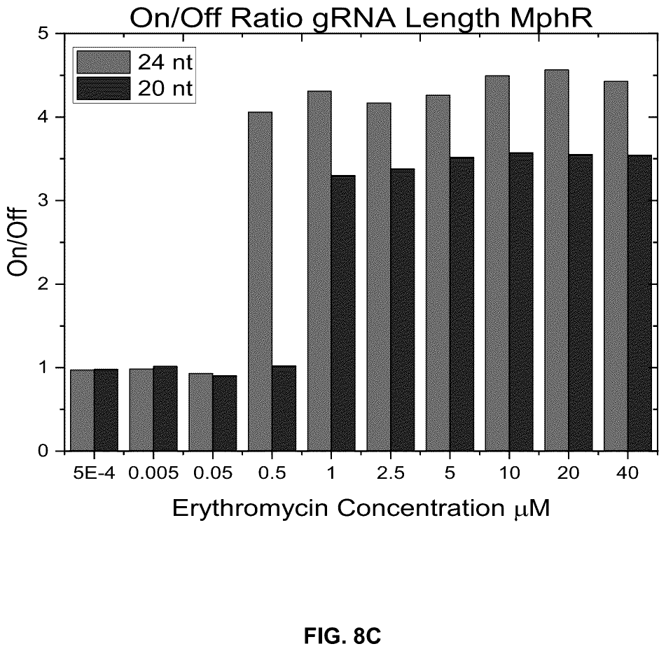

Another potential control parameter lies with the gRNA. Making modifications to the gRNA is relatively easy and could potentially impact interactions with the target DNA. Although it is generally thought that only the first 20 nucleotides of the spacer sequence in the gRNA play a role in Cas12a interactions and the remaining complementary region does not aid in target recognition (29, 31, 32), one study has shown that a target length of 24 nt enhanced trans-cleavage activity on single-stranded DNA33. Thus, gRNAs with different spacer lengths were tested to determine their effect on small molecule detection. gRNA with spacer lengths of 17, 20, and 24 nts were evaluated with a range of aTc and Doxy concentrations. Reducing the gRNA spacer length resulted in quicker trans-cleavage activity, but also increased overall leakage (FIG. 8). On/off ratios were calculated for the different lengths by taking the ratio of the small molecule positive sample to the small molecule negative sample (FIG. 8A). The 20-nt spacer resulted in much higher on/off ratios throughout a range of aTc concentrations and was used to repeat the 50-fold tetR dimer:DNA experiments with 5 nM and 50 nM DNA. In contrast, MphR provided strong signals and higher on/off ratios using 24-nt gRNA spacer length (FIG. 8B).

FIG. 7C shows the results for the 5 nM and 50 nM DNA concentrations at 30 minutes of cleavage using the 20-nt spacer. The EC 50 for aTc detection did not change but the same results were obtained in half the time (Table 7). The EC 50 for Doxy detection with 5 nM DNA drastically shifted to 0.0004 μM in 30 minutes (Table 8). A slight shift was also seen for the 50 nM set, going from 1.47 μM to 0.65 μM by reducing the guide length. Reducing the gRNA length presented a means to further sensitize and speed up the Cas12a-driven reaction.

Detection limits were also considered using the standard definition of LOD which considers LOD to be equal to the fluorescence of the blank sample plus three times the standard deviation (Table 7 and Table 8). The detection limit was determined using the equation (39):

Sₘ=S̅_{bl}+kS_{bl}

Here, S̅_{bl} is equal to the mean of the SM negative samples and kS_{bl} is the number of standard deviations of the negative sample added. For the Doxy experiments, 8 replicates were run and k=3. For aTc experiments, 10 replicates were run and k 32 3. The detection limit (bolded) was then determined as the lowest concentration corresponding to a fluorescence signal great than Sₘ (the mean of small molecule negative samples+3× the standard deviation).

TABLE 7
Detection Limit Determination of aTc and Doxy
for TetR biosensor using 20 nt guide.
20 nt gRNA Doxy Detection: Sₘ = 8,996
Detection Limit = 4 nM

| Concentration | Fluorescence | Standard |
|---|---|---|
| Doxy | Mean | Deviation |
| 0 | 6698.37 | 766.38 |
| 0.004 | 9522.25 | 415.7 |
| 0.04 | 40251.25 | 2817.1 |
| 0.4 | 41099.25 | 2883.72 |
| 1 | 42083 | 2854.57 |

20 nt gRNA aTc Detection: Sₘ= 26,537.68
Detection Limit = 100 nM

| Concentration | Fluorescence | Standard |
|---|---|---|
| aTc | Mean | Deviation |
| 0 | 21167.6 | 1790.02 |
| 0.005 | 21303.25 | 1494.8 |
| 0.01 | 21302 | 1817.53 |
| 0.05 | 24918.25 | 3064.1998 |
| 0.1 | 32806 | 1632.29 |
| 1 | 40622.5 | 3693.42 |

TABLE 8
Detection Limit Determination of aTc and Doxy
for TetR biosensor using 24 nt guide.
24 nt gRNA Doxy Detection: Sₘ = 6,810.91
Detection limit = 40 nM

| Concentration | Fluorescence | Standard |
|---|---|---|
| Doxy | Mean | Deviation |
| 0 | 6466.87 | 114.68 |
| 0.004 | 6458.5 | 151.422 |
| 0.04 | 9054.5 | 345.82 |
| 0.4 | 32942 | 2239.65 |
| 1 | 33837 | 1757.22 |

24 nt gRNA aTc Detection: Sₘ = 11,121.9
Detection limit = 50 nM

| Concentration | Fluorescence | Standard |
|---|---|---|
| aTc | Mean | Deviation |
| 0 | 8857.5 | 754.8 |
| 0.005 | 9199.5 | 358.49 |
| 0.01 | 8795 | 576.47 |
| 0.05 | 12129.5 | 796.16 |
| 0.1 | 20059 | 1406.3 |
| 1 | 30309.5 | 3791 |

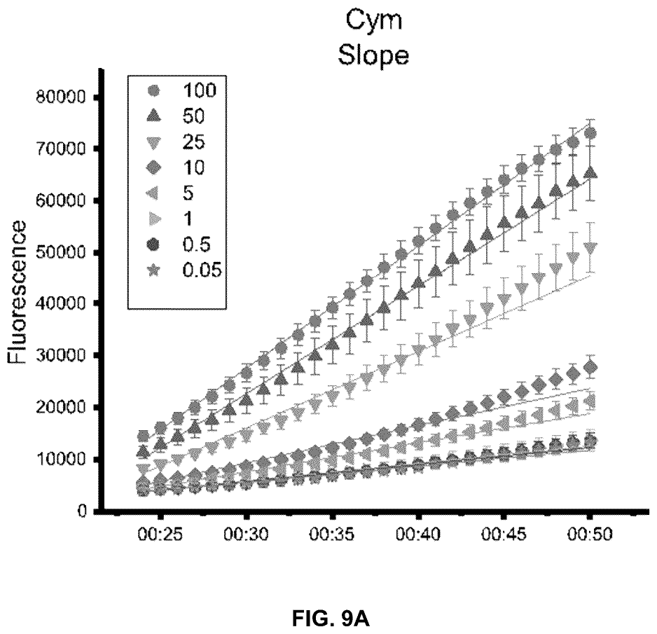

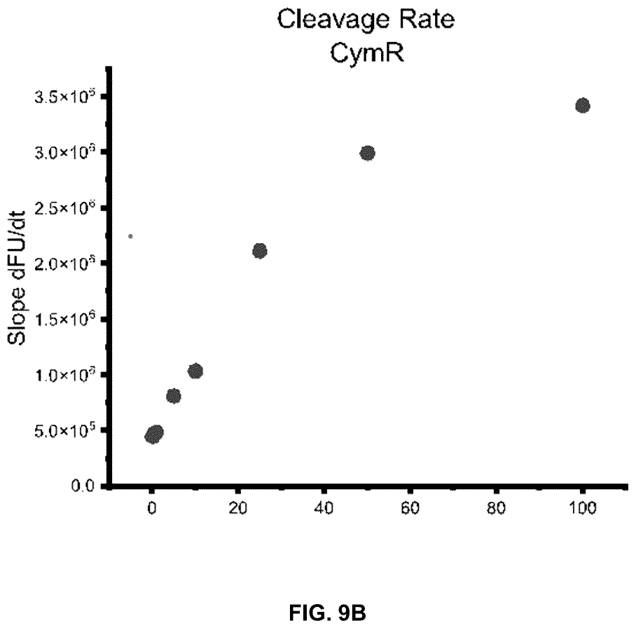

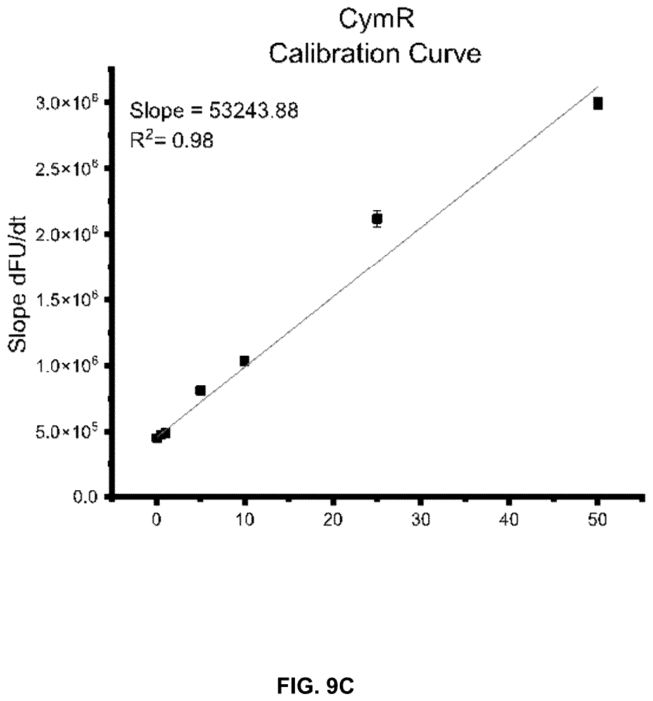

Using this definition, the limit of detection for the TetR biosensor using a 24-nt gRNA was 40 nM for doxycycline and 50 nM for aTc. This is similar to the limit of detection determined using the EC 50 values for both SM. For the 20 nt gRNA experiments, the LOD for aTc was 100 nM, and 4 nM for doxycycline. This showed some variation in comparison to the EC50 values. Considering the EC 50 value for doxycycline detection using 20 nt gRNA was resolved to be lower than the lowest end of SM experimentally tested, the standard definition of LOD is found to be more appropriate and it can be concluded that the LOD ranges between 0.4 and 4 nM. The possibility of using the biosensors for quantitative detection of analytes by measuring the fluorescence generated over time by cleavage as a function of analyte concentration was also examined. These experiments revealed that the fluorescence difference as a function of analyte concentration is sufficient to allow quantitative detection for the assays (FIG. 9A-C).

### Example 5—Incorporating Paper-Based Detection using Lateral Flow Devices

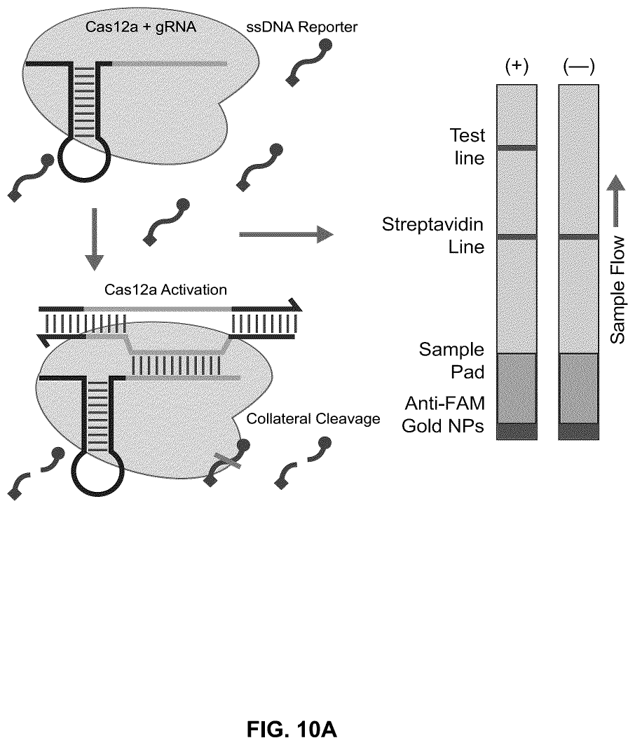

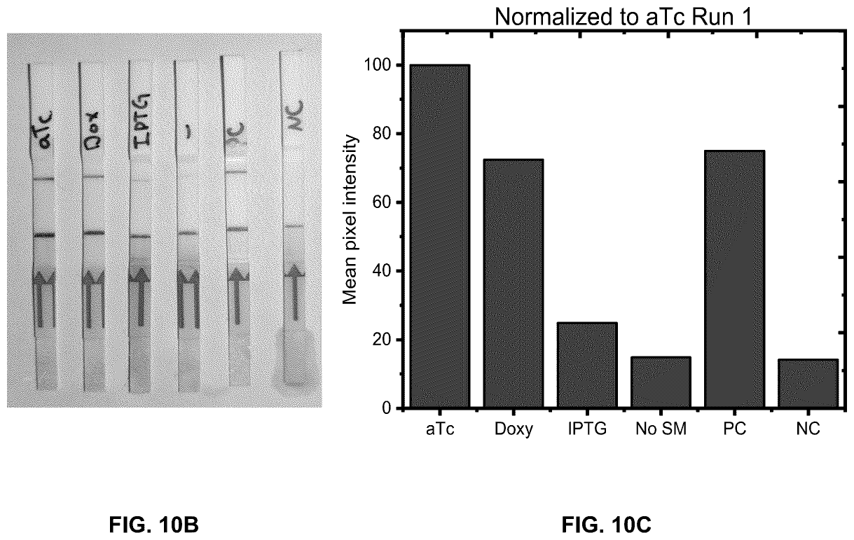

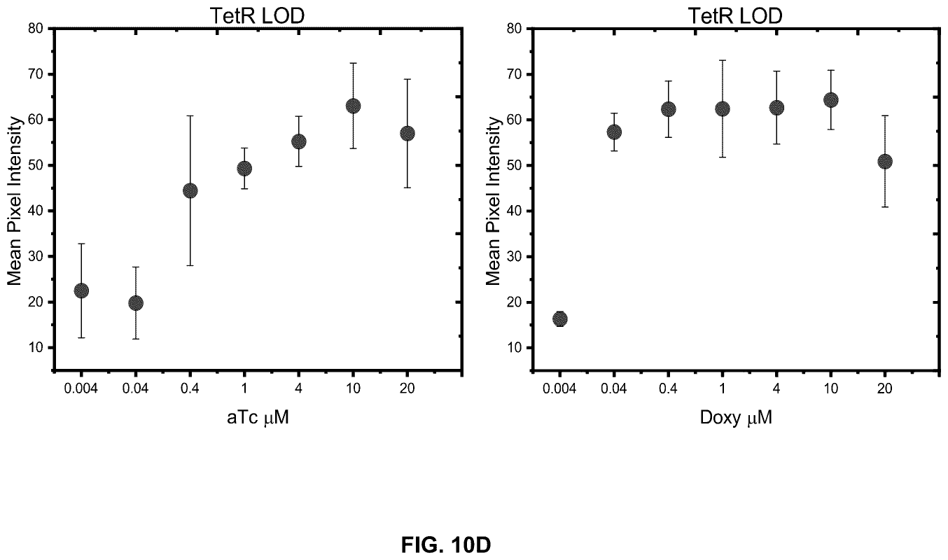

The trans-cleavage capabilities of Cas12a can cleave any single-stranded DNA in the system, enabling a variety of chemical modifications to be used in the single-stranded reporter. This feature allows for implementation of a visible paper-based lateral flow readout system using reporters with FAM and biotin modifications on either end (19, 21-26). When paired with a lateral flow assay, the fully intact reporter binds to the first line (control line) along with an attached gold nanoparticle/antibody complex, and can be visualized, as shown in FIG. 10. When Cas12a enzyme is active, the reporter is cleaved and the portion containing the gold nanoparticle flows down the assay and can be visualized by the formation of the second band. Thus, the presence of two bands is used to indicate the presence of the target small molecule.

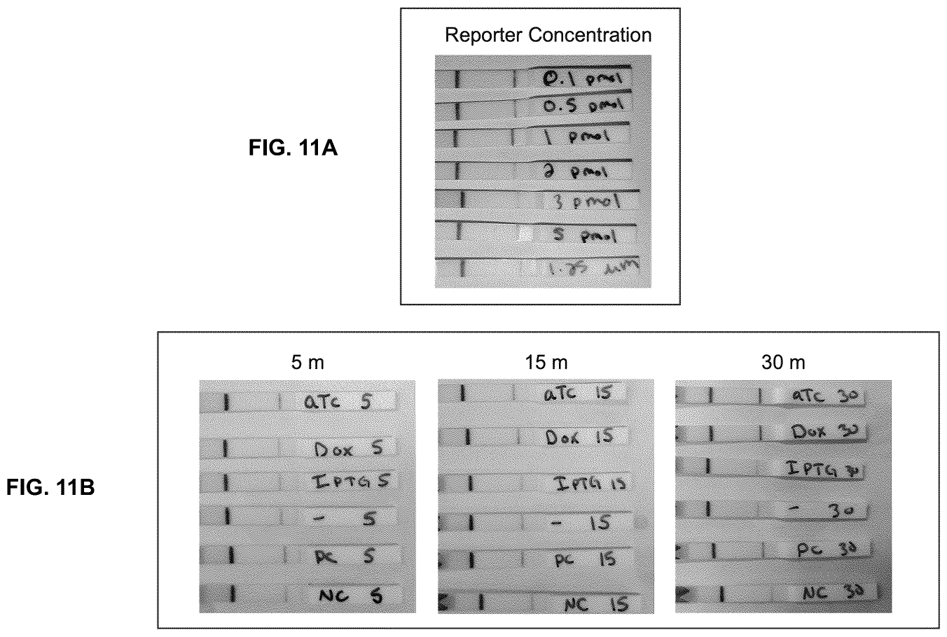

The lateral flow readout system was tested and optimized using the TetR system. Prior to this, different reporter concentrations were added to the lateral flow assay with only running buffer to find the reporter concentration that provided the lowest background signal for the test band. Optimizing this step is critical because the reporter concentration must be high enough to work at the K_{d} of the reporter and gold nanoparticle/antibody complex, ensuring the reporter binds to the complex, and low enough to not see leakage or flowthrough onto the test line. This behavior is caused by the Hooke effect, where too high of concentration can cause unbound nanoparticle/antibodies to flow directly to the test line. The lowest leakage was seen between 2-3 pmol of reporter, which corresponds to a reporter concentration of 200-300 nM in the lateral flow running buffer (FIG. 11A).

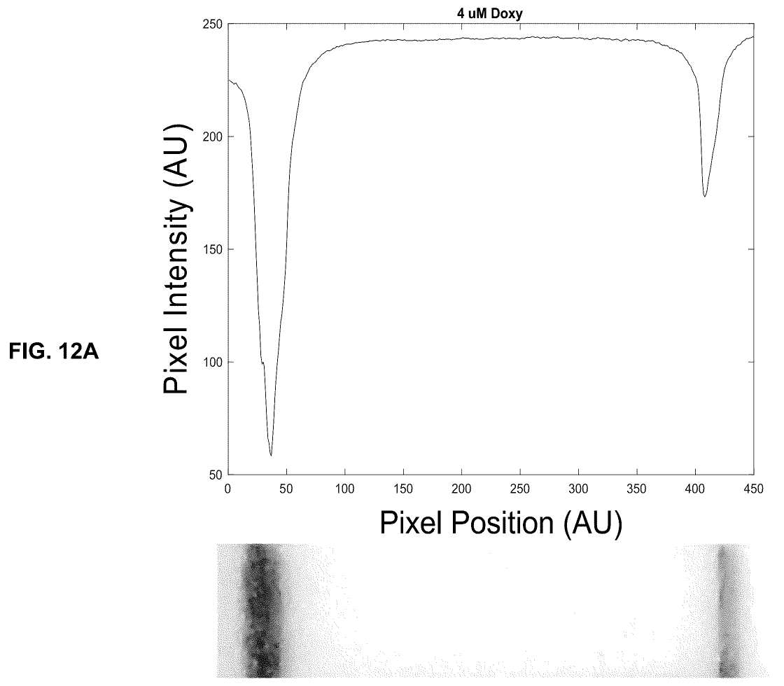

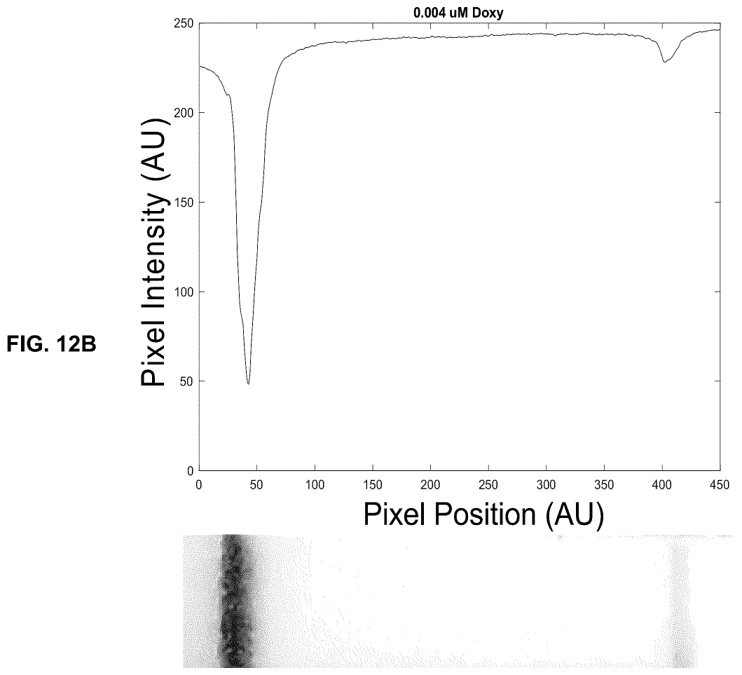

Next, the lateral flow readout was tested by introducing aTc, Doxy, and IPTG to the TetR biosensor. The samples were incubated with Cas12a cleavage mixture containing the 20-nt gRNA. Different cleavage times were tested but results showed cleavage required 30 minutes to provide strong band signals (FIG. 11B). FIG. 10B shows a strong second band present on samples containing aTc and Doxy with little to no banding observed for samples containing IPTG or no analyte. Thus, the lateral flow assay can provide a visual readout mechanism for the biosensor, and the assay remains small-molecule specific. These results were confirmed through a quantitative analysis of the lateral flow assays performed by measuring the band intensities from photographs of the lateral flow strips (FIG. 10C). The plotting of pixel intensity for quantitative interpretation is shown in FIG. 12.

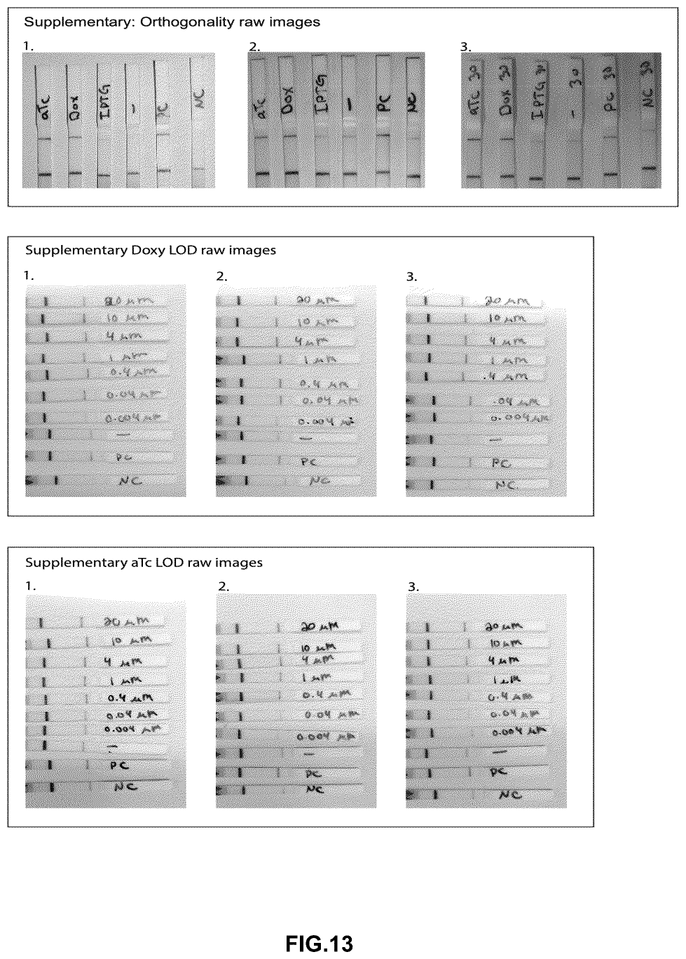

To ensure the biosensor could provide adequate sensitivity, limit of detection experiments were repeated using TetR for detecting aTc and Doxy on the lateral flow assays. FIG. 10D shows the mean band intensities plotted over three replicates for both aTc and Doxy. At 30 minutes of cleavage, aTc shows a discernible signal at 400 nM with a calculated EC 50 of 380 nM. Doxy again proved more sensitive to detection with a steep signal loss between 40 and 4 nM and an EC 50 value down to 27 nM. Replicates of the lateral flow assays are provided in FIG. 13.

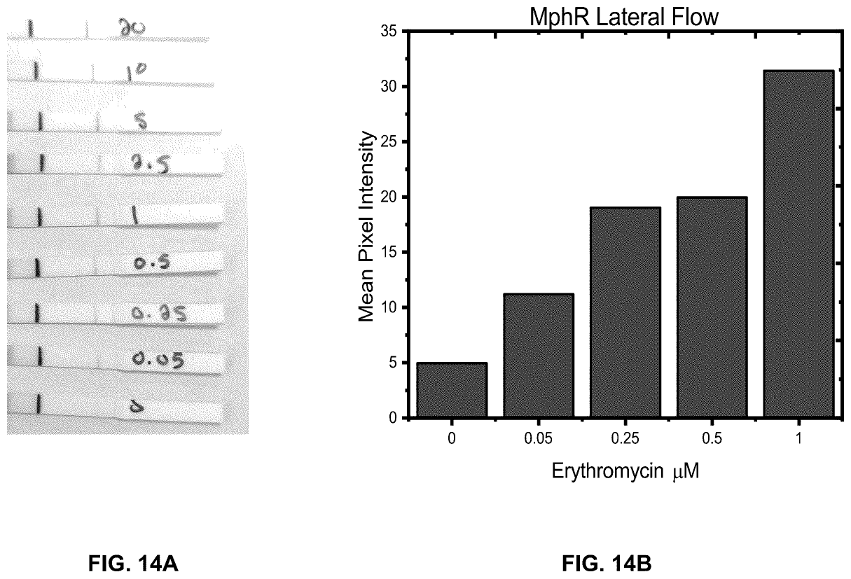

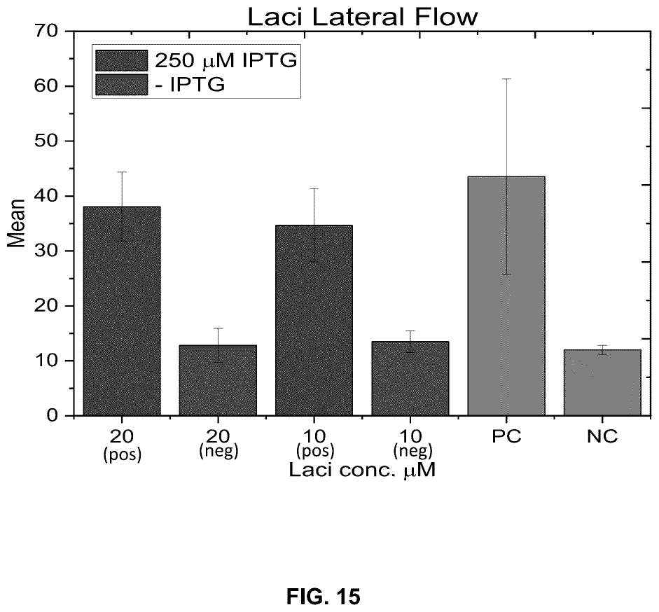

The robustness of the lateral flow readout scheme was further tested using the TF MphR to detect the antibiotic erythromycin. FIG. 14A shows the results of lateral flow testing after after 15 minutes of cleavage, with signal going down below 1 μM Erythromycin, and disappearing completely with no small molecule present. A closer look at the mean pixel intensity of the bands for the assays for 1, 0.5, 0.25, 0.05 μM and no SM confirm a decrease below 1 μM but show a readable signal in the ˜100 nM range (FIG. 14B). Lateral flow readout was also tested using the LacI-IPTG biosensor. A higher ratio between the IPTG+ and − samples were seen even with high concentrations of Lad, but a discernible difference in SM+ samples was still visualized (FIG. 15).

### Example 6—Biosensor Functionality in Water Samples

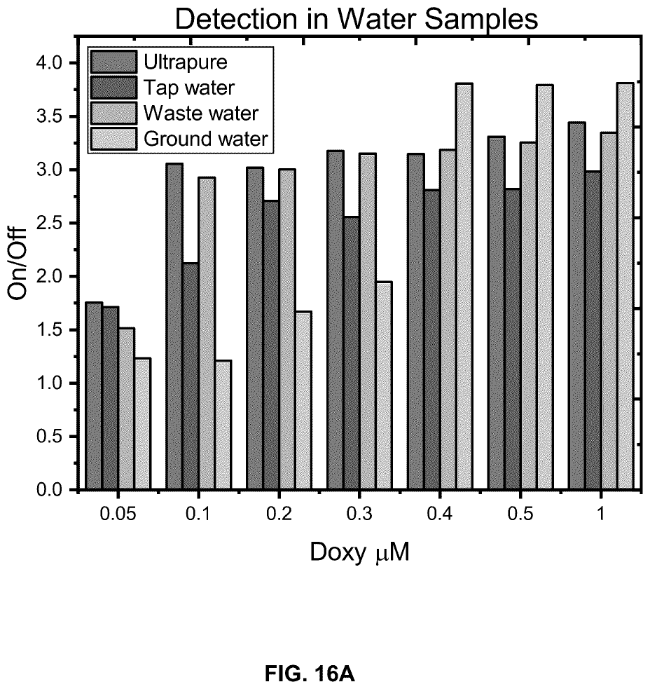

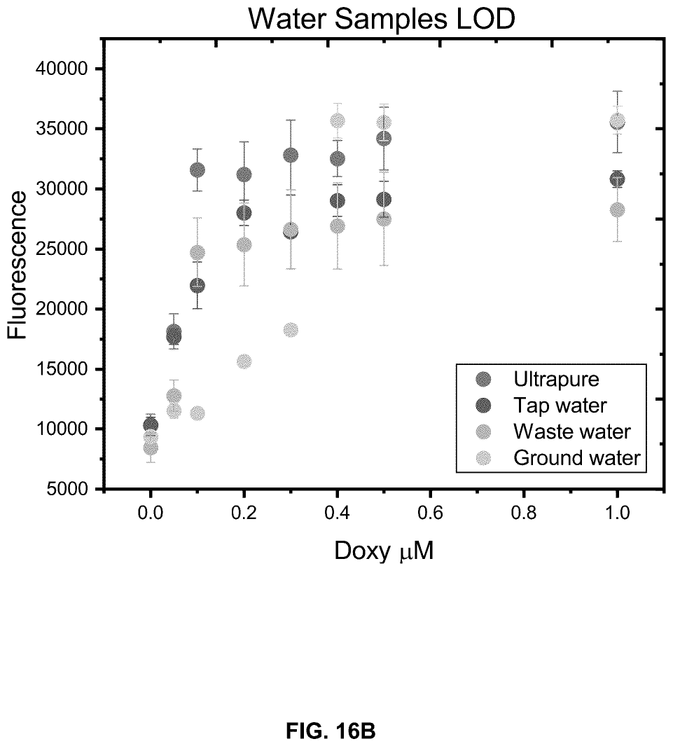

This biosensor can be applied to the detection of antibiotics in water samples with the detection ranges for doxycycline found ranging from high pM to ˜100 nM. FIGS. 16A and B show that the biosensor can still operate in different water sources of tap water, ground water, and wastewater for the detection of doxycycline. All samples show high on/off ratios at 100 nM with the exception of groundwater which is in the 10's of ppb range required for antibiotic detection. Although the on/off ratios for ground water were weaker at 100 nM, there was still a fluorescence signal 3 times the standard deviation of the negative control sample even at 50 nM in ground water for the detection of Doxy. These results show this biosensor can work in the necessary concentration range for antibiotic detection in water sources stated in Danner et al. (15).

### Example 7—Exemlary forms of biosensor devices

In some embodiments, the dsTarget complexes are incubated with the corresponding transcription factors in incubation buffer. After incubation, reporter constructs are added and the reaction mixture is incubated again. Next, the DNA/TF mixture is added to a Cas cleavage reaction, comprising Cas nuclease and appropriate buffers. In some embodiments, each incubation step can take place in different vessels. For example, in some embodiments, the steps can be completed in different wells of a multi-well plate. In some embodiments, the substrates, the binding proteins, the reporter constructs, the Cas nuclease, and the gRNAs are lyophilized and added to appropriate buffers for reconstitution before or during the method. In some embodiments, the Cas cleavage reaction is added to a microplate and then imaged with a fluorescent plate reader. In other embodiments, the Cas cleavage reaction is added directly to a dipstick, allowed to incubate for a short period of time, and then imaged with a camera such as a cell phone camera. There are many possible reporter constructs, including but not limited to small molecule reporters, enzymatic reporters, fluorescent or chemiluminescent reporters, or fluorogenic aptamer-dyes.

### REFERENCES

1. Cao, J. et al. Harnessing a previously unidentified capability of bacterial allosteric transcription factors for sensing diverse small molecules in vitro. Sci. Adv. 4, 1-12 (2018).
2. Rodriguez-Serrano, A. F. & Hsing, I. M. Allosteric Regulation of DNA Circuits Enables Minimal and Rapid Biosensors of Small Molecules. ACS Synth. Biol. 10, 371-378 (2021).
3. Jung, J. K. et al. Cell-free biosensors for rapid detection of water contaminants. Nat. Biotechnol. 38, 1451-1459 (2020).
4. Yao, Y. et al. Development of small molecule biosensors by coupling the recognition of the bacterial allosteric transcription factor with isothermal strand displacement amplification. Chem. Commun. 54, 4774-4777 (2018).
5. Jung, J. K., Archuleta, C. M., Alam, K. K. & Lucks, J. B. Programming cell-free biosensors with DNA strand displacement circuits. Nat. Chem. Biol. (2022) doi:10.1038/s41589-021-00962-9.
6. Wan, X. et al. Cascaded amplifying circuits enable ultrasensitive cellular sensors for toxic metals. Nat. Chem. Biol. 15, 540-548 (2019).
7. Meyer, A. J., Segall-Shapiro, T. H., Glassey, E., Zhang, J. & Voigt, C. A. Escherichia coli “Marionette” strains with 12 highly optimized small-molecule sensors. Nat. Chem. Biol. 15, 196-204 (2019).
8. Liang, M. et al. A CRISPR-Cas12a-derived biosensing platform for the highly sensitive detection of diverse small molecules. Nat. Commun. 10, (2019).
9. Kim, H., Lee, S., Yoon, J., Song, J. & Park, H. G. CRISPR/Cas12a collateral cleavage activity for simple and rapid detection of protein/small molecule interaction. Biosens. Bioelectron. 194, 113587 (2021).
10. Xiong, Y. et al. Functional DNA Regulated CRISPR-Cas12a Sensors for Point-of-Care Diagnostics of Non-Nucleic-Acid Targets. J. Am. Chem. Soc. 142, 207-213 (2020).
11. Niu, C. et al. Aptamer assisted CRISPR-Cas12a strategy for small molecule diagnostics. Biosens. Bioelectron. 183, 113196 (2021).
12. Peng, L. et al. CRISPR-Cas12a based aptasensor for sensitive and selective ATP detection. Sensors Actuators, B Chem. 320, 128164 (2020).
13. Iwasaki, R. S. & Batey, R. T. SPRINT: A Cas13a-based platform for detection of small molecules. Nucleic Acids Res. 48, 1-16 (2020).
14. Gothwal, R. & Shashidhar, T. Antibiotic Pollution in the Environment: A Review. Clean-Soil, Air, Water 43, 479-489 (2015).
15. Danner, M. C., Robertson, A., Behrends, V. & Reiss, J. Antibiotic pollution in surface fresh waters: Occurrence and effects. Sci. Total Environ. 664, 793-804 (2019).
16. Myhrvold, C. et al. Field-deployable viral diagnostics using CRISPR-Cas13. http://science.sciencemag.org/.
17. Chen, J. S. et al. CRISPR-Cas12a target binding unleashes indiscriminate single-stranded DNase activity. Science (436-439). 360, (2018).
18. Gootenberg, J. S. et al. Nucleic acid detection with CRISPR-Cas13a/C2c2. Science 356, 438-442 (2017).
19. Zhang, F., Abudayyeh, O. O., Gootenberg, J. S., Sciences, C. & Mathers, L. A protocol for detection of COVID-19 using CRISPR diagnostics. Bioarchive 1-8 (2020).
20. Zhang, Y. & Ying, J. Y. Homogeneous Immunochemical Assay on the Lateral Flow Strip for Measurement of DNase i Activity. Anal. Chem. 87, 10193-10198 (2015).
21. Abudayyeh, O. O., Gootenberg, J. S., Kellner, M. J. & Zhang, F. Nucleic Acid Detection of Plant Genes Using CRISPR-Cas13. Cris. J. 2, 165-171 (2019).
22. Metsky, H. C., Freije, C. A., Kosoko-Thoroddsen, T. S. F., Sabeti, P. C. & Myhrvold, C. CRISPR-based surveillance for COVID-19 using genomically-comprehensive machine learning design. bioRxiv 1-11 (2020) doi:10.1101/2020.02.26.967026.
23. Tsou, J. H., Leng, Q. & Jiang, F. A CRISPR Test for Detection of Circulating Nuclei Acids. Transl. Oncol. 12, 1566-1573 (2019).
24. Sullivan, T. J., Dhar, A. K., Cruz-Flores, R. & Bodnar, A. G. Rapid, CRISPR-Based, Field-Deployable Detection Of White Spot Syndrome Virus In Shrimp. Sci. Rep. 9, 1-7 (2019).
25. Broughton, J. P. et al. CRISPR-Cas12-based detection of SARS-CoV-2. Nat. Biotechnol. 38, 870-874 (2020).
26. Zhang, Y. mu, Zhang, Y. & Xie, K. Evaluation of CRISPR/Cas12a-based DNA detection for fast pathogen diagnosis and GMO test in rice. Mol. Breed. 40, (2020).
27. Kamionka, A., Bogdanska-Urbaniak, J., Scholz, O. & Hillen, W. Two mutations in the tetracycline repressor change the inducer anhydrotetracycline to a corepressor. Nucleic Acids Res. 32, 842-847 (2004).
28. Singh, D. et al. Real-time observation of DNA target interrogation and product release by the RNA-guided endonuclease CRISPR Cpf1 (Cas12a). Proc. Natl. Acad. Sci. 115, 5444-5449 (2018).
29. Strohkendl, I., Saifuddin, F. A., Rybarski, J. R., Finkelstein, I. J. & Russell, R. Kinetic Basis for DNA Target Specificity of CRISPR-Cas12a. Mol. Cell 71, 816-824.e3 (2018).
30. Cheng, Y. et al. Comparative study between macrolide regulatory proteins MphR(A) and MphR(E) in ligand identification and DNA binding based on the rapid in vitro detection system. Anal. Bioanal. Chem. 408, 1623-1631 (2016).
31. Yamano, T. et al. Crystal Structure of Cpf1 in Complex with Guide RNA and Target DNA. Cell 165, 949-962 (2016).
32. Gao, P., Yang, H., Raj ashankar, K. R., Huang, Z. & Patel, D. J. Type v CRISPR-Cas Cpf1 endonuclease employs a unique mechanism for crRNA-mediated target DNA recognition. Cell Res. 26, 901-913 (2016).
33. Fuchs, R. T., Curcuru, J., Mabuchi, M., Yourik, P. & Robb, G. B. Cas12a trans-cleavage can be modulated in vitro and is active on ssDNA, dsDNA, and RNA. bioRxiv 21-26 (2019) doi:10.1101/600890.
34. Li, S. et al. A platform for the development of novel biosensors by configuring allosteric transcription factor recognition with amplified luminescent proximity homogeneous assays. Chem. Commun. 53, 99-102 (2017).
35. Manyi-Loh, C., Mamphweli, S., Meyer, E. & Okoh, A. Antibiotic use in agriculture and its consequential resistance in environmental sources: Potential public health implications. Molecules vol. 23 (2018).
36. Scarano, C. et al. Antibiotic resistance of Aeromonas ssp. strains isolated from Sparus aurata reared in Italian mariculture farms. Int. J. Food Microbiol. 284, 91-97 (2018).
37. Pinheiro, A., Rosa Albano, R. M., Alves, T. C., Kaufmann, V. & Da Silva, M. R. Veterinary antibiotics and hormones in water from application of pig slurry to soil. Agric. Water Manag. 129, 1-8 (2013).
38. Carina C Clingman, Laura M Deveau, Samantha A Hay, Ryan M Genga, Shivender MD Shandilya, Francesca Massi, Sean P Ryder (2014) Allosteric inhibition of a stem cell RNA-binding protein by an intermediary metabolite eLife 3:e02848.
39. Skoog, D, A., Holler, F. J. & Crouch, S. R. Principles of Instrumental Analysis. (2007).

We claim:

1. A method of detecting a target molecule in a sample, the method comprising:
(a) contacting to the sample
a synthetic double-stranded DNA (dsDNA) substrate comprising an operator sequence specific to a binding protein and a protospacer adjacent motif (PAM) sequence;
the binding protein;
a Cas nuclease that exhibits collateral single-stranded DNase (ssDNase) activity;
a guide RNA (gRNA), wherein the gRNA hybridizes the reverse complement of the operator sequence or the operator sequence; and
a single-stranded DNA (ssDNA) reporter construct comprising a detectable tag,
wherein, in the presence of the target molecule, the target molecule binds to the binding protein, the gRNA forms a complex with the PAM sequence, and the Cas nuclease cleaves the ssDNA reporter construct; and
(b) detecting cleavage of the ssDNA reporter construct, thereby detecting the target molecule in the sample.

2. The method of claim 1, wherein the binding protein is a transcription factor.

3. The method of claim 2, wherein the transcription factor is selected from TetR, PhiF, CymR, LacI, MphR, and BetL.

4. The method of claim 1, wherein the Cas nuclease is selected from Cas12a, Cas13a, Cas13b, Cas13d, Cas12g1, and Cas12i1.

5. The method of claim 4, wherein the Cas nuclease is Cas 12a, and wherein the Cas12a is from the Lachnospiraceae family.

6. The method of claim 1, wherein the detectable tag comprises biotin and one of digoxigenin and FAM (fluorescein).

7. The method of claim 1, wherein the target molecule is an antibiotic.

8. The method of claim 1, wherein the target molecule is selected from aTc, doxycycline, DAPG, cuminic acid, IPTG, clarithromycin, erythromycin, azithromycin, and roxithromycin.

9. The method of claim 1, wherein the sample is a water sample.

10. A biosensor device comprising:
a plurality of synthetic dsDNA substrates, each substrate comprising an operator sequence specific to a different binding protein, and a protospacer adjacent motif (PAM) sequence;
a plurality of binding proteins, wherein each binding protein binds to one of the operator sequences;
a ssDNA reporter construct comprising a detectable tag;
a Cas nuclease that exhibits collateral ssDNase activity; and
a plurality of gRNAs, wherein each gRNA hybridizes the reverse complement of the operator sequence or the operator sequence.

11. The biosensor device of claim 10, wherein the biosensor device is selected from a lateral flow test device and a microplate.

12. The biosensor device of claim 11, wherein the biosensor device is a microplate comprising a plurality of wells, and
wherein for each of the plurality of synthetic dsDNA substrates,
the substrate, the binding protein that binds to the substrate, and the gRNA which hybridizes the reverse complement of the operator sequence or the operator sequence are provided in a separate well.

13. The biosensor device of claim 11, wherein the biosensor device is a lateral flow test device, and wherein the detectable tag comprises biotin and one of FAM and digoxigenin.

14. The biosensor device of claim 12, wherein the detectable tag is a fluorophore-quencher pair.

15. The biosensor device of claim 10, wherein each of the binding proteins is a transcription factor.

16. The biosensor device of claim 15, wherein the transcription factor is selected from TetR, PhiF, CymR, LacI, MphR, and BetL.

17. The biosensor device of claim 10, wherein the Cas nuclease is Cas12a.

18. The biosensor device of claim 17, wherein the Cas12a is from the Lachnospiraceae family.

19. The biosensor device of claim 10, wherein the substrates, the binding proteins, the reporter constructs, the Cas nuclease, and the gRNAs are lyophilized.

20. A kit comprising the biosensor device of claim 10 and instructions for use.
